(19) United States
(12) Patent Application Publication
(10) Pub. No.: US 2016/0038632 A1
(43) Pub. Date: Feb. 11, 2016

(54) Adaptive Drug Delivery from an Artificial Polymer Skin with Tunable Properties for Tissue Engineering

(71) Applicant: Massachusetts Instutite of Technology, Cambridge, MA (US)

(72) Inventors: Nisarg Jaydeep Shah, Cambridge, MA (US); Md. Nasim Hyder, Somerville, MA (US); Paula Therese Hammond, Newton, MA (US)

(21) Appl. No.: 14/454,504

(22) Filed: Aug. 7, 2014

(51) Int. Cl. A61L 27/18; A61L 27/58; A61L 27/54; A61L 27/56

(52) U.S. Cl. CPC A61L 27/18; A61L 27/56; A61L 27/58; A61L 27/54; A61L 2300/252; A61L 2300/414; A61L 2300/602; A61L 2430/02; A61L 2430/20; A61L 2430/34

(57) ABSTRACT

The present invention provides, among other things, a composite device comprised of a porous polymer membrane carrying active growth factors. Composite devices are characterized by an ability to controllably degrade for repair of bone and/or tissue defects sustained from traumatic wounds or congenital defects through eluting growth factor over readily adapted time scales inducing a natural wound healing cascade and rapid bone repair. Methods of making and using provided devices are also disclosed.

### GOVERNMENT SUPPORT

This invention was made with government support under Grant/Contract No. 5R01EB010246 with O.S.P. Project No. 6920630, and Grant/Contract No. 5R01AG029601 with O.S.P. Project No. 6914977 from the National Institutes of Health. The government has certain rights in the invention.

### BACKGROUND OF THE INVENTION

A variety of materials and technologies have been developed to promote or achieve bone and/or tissue regeneration and/or restoration. Both natural and synthetic materials have been utilized; each has certain advantages and disadvantages.

### SUMMARY OF THE INVENTION

The present invention provides, among other things, composite devices for promoting bone and/or tissue regeneration and methods relating to creating such composite devices. In some embodiments, the present invention demonstrates that rapid repair of large bone and/or tissue defects is achievable without complex implant surgery and/or autograft bone.

In some embodiments, the present invention recapitulates aspects of the natural healing cascade and provides targeted delivery of growth factor to bone and/or tissue defects within an area of a body. In some embodiments, targeted delivery of growth factor is localized to an area of a defect within a body so that a risk of unwanted additional biological affects and/or toxicity is minimized.

In some embodiments, the present invention provides a degradable composite capable of controlled delivery of growth factor. In some embodiments, devices and methods described herein include or comprise biologically degradable composites that carry and release growth factor over adaptable time scales to induce rapid repair of bone and/or tissue by initiating and sustaining a defect healing cascade.

In some embodiments, a provided device is or comprises a porous polymer membrane.

In some embodiments, a porous polymer membrane is utilized to enclose, cover, fill, and/or isolate a defect. In some embodiments, a porous polymer membrane and/or a composite device is conformable, for example so that its shape can adapt to a defect. In some embodiments, a porous polymer membrane and/or a device may be sized, for example, by cutting or molding so that its shape fits a defect.

In some embodiments, a defect is large. In some embodiments, a defect size is of critical size, such that it does not naturally heal. In some embodiments, a defect is a large bone defect. In some embodiments, a bone defect is or comprises a craniomaxillofacial (CMF) defect. In some embodiments, a bone defect is or comprises a segmental bone defect. In some embodiments, a defect is or comprises an augmentation for a dental implant. In some embodiments, a defect is or comprises a neural tissue defect. In some embodiments, a defect is a vascular tissue defect.

In some embodiments, closure of a large defect initiates within a week of placement of a composite device.

Among other things, the present invention provides compositions and methods for assembly of composite devices. In some embodiments, a composite device includes a porous polymer membrane associated with at least one drug and/or growth factor, wherein a membrane degrades, decomposes, and/or delaminates to release at least one growth factor. In some embodiments, a composite device includes a porous polymer membrane that is both conjugated to a bisphosphonate and associated with at least one drug and/or growth factor, wherein a membrane degrades to release at least one growth factor. In some embodiments, a device is characterized in that at least one growth factor releases having a staggered release. In some embodiments, a device is characterized in that at least one growth factor releases having a concurrent release.

In some embodiments, a porous polymer membrane of a composite device is comprised of poly(glycolide-colactide) copolymer (PLGA)/polylactic acid (PLA). In some embodiments, mechanical properties of a porous polymer membrane are a function of a ratio of PLA to PGLA. In some embodiments, flexibility of a porous polymer membrane is a function of a ratio of PLA to PGLA. In some embodiments, a lower PLA to PGLA ratio results in a more elastic porous polymer membrane. In some embodiments, degradation of a porous polymer membrane is a function of a ratio of PLA to PGLA. In some embodiments, a lower PLA to PGLA ratio results in a porous polymer membrane having a faster degradation rate. In some embodiments, a 50:50 ratio of PLA to PGLA yielded a degradation half-life of about four weeks for a cranial defect healing. In some embodiments, a porous polymer membrane is comprised of polycaprolactone (PCL). In some embodiments, a PCL membrane degrades, decomposes, and or delaminates to yield a half-life of up to one year for cranial defect healing.

In some embodiments, a porous polymer membrane comprises a bisphosphonate conjugated to PLGA. In some embodiments, a bisphosphonate is alendronate.

In some embodiments, a porous polymer membrane comprises a small molecule. In some embodiments, a porous polymer membrane degrades releasing a small molecule. In some embodiments, a small molecule is gentamicin.

In some embodiments, a porous polymer membrane is biocompatible, biodegradable, and/or resorbable and has a thickness of at least about 5 microns to at least about 200 microns.

In some embodiments, a porous polymer membrane is characterized by a plurality of interconnected pores. In some embodiments, a pore size of a plurality of pores varies between a top surface of a porous polymer membrane and a bottom surface of a porous polymer membrane. In some embodiments, the porous polymer membrane has non-uniform porosity. In some embodiments, a pore size of a plurality of pores increases between a top surface of a porous polymer membrane and a bottom surface of a porous polymer membrane. In some embodiments, a pore size of a plurality of pores varies from 100 nanometers on a top surface of a porous polymer membrane to at least about 1 mm on a bottom surface of a porous polymer membrane. In some embodiments, a pore size of a plurality of pores varies from 200 nanometers on a top surface of a porous polymer membrane to 2 mm on a bottom surface of a porous polymer membrane. In some embodiments, a pore size is uniform throughout a porous polymer membrane. In some embodiment, the porous polymer membrane has uniform porosity.

In some embodiments, a top surface of a porous polymer membrane is permeable and a bottom surface of a porous polymer membrane is impermeable.

In some embodiments, a composite device further comprises a multilayer film. In some embodiments, a composite device is or comprises a layer-by-layer (LbL) film. In some embodiments, an LbL film is associated with a porous polymer membrane. In some embodiments, an LbL film is conformally coated on a top surface of a porous polymer membrane. In some embodiments, an LbL film is hydrolytically degradable. In some embodiments, an LbL film has a thickness of at least about 100 nanometers to at least about 1 micron. In some embodiments, an LbL film comprises alternating polyelectrolyte multilayers (PEM), wherein adjacent layers of a multilayer film are associated with one another via one or more non-covalent interactions.

In some embodiments, a multilayer LbL film comprises at least one growth factor. In some embodiments, a porous polymer membrane with an LbL film associated with a porous polymer membrane each comprise at least one growth factor. In some embodiments, an LbL film comprising growth factor is associated with a porous polymer membrane without growth factor. In some embodiments, a multilayer LbL film degrades, decomposes, and/or delaminates to release at least one growth factor. In some embodiments, an LbL film is physically sequestered within the interconnected pore structure of the membrane, resulting in a differential rate of release. In some embodiments, a device is characterized in that at least one growth factor releases from a porous polymer membrane and/or an LbL film having a staggered release. In some embodiments, a device is characterized in that at least one growth factor releases from a porous polymer membrane and/or an LbL film having a concurrent release.

In some embodiments, growth factor is associated within alternating PEM of an LbL film. In some embodiments, at least one growth factor is associated within an alternating PEM of an LbL film. In some embodiments, at least two growth factors are associated within an alternating PEM of an LbL film. In some embodiments, at least three growth factors are associated within an alternating PEM of an LbL film. In some embodiments, at least four growth factors are associated within an alternating PEM of an LbL film. In some embodiments, a loading dose of growth factor is at least about 10 nanograms to at least about 10 micrograms. In some embodiments, a loading dose of growth factor is at least about 10 nanograms to at least about 10 micrograms. In some embodiments, a loading dose of growth factor is at least about 10 nanograms to at least about 10 micrograms. In some embodiments, a loading dose of growth factor is at least about 40 micrograms. In some embodiments, a loading dose of growth factor is at least about 10 micrograms. In some embodiments, a loading dose of growth factor is at least about 50 micrograms. In some embodiments, a loading dose of growth factor is at least about 100 micrograms. In some embodiments, growth factor is a bone morphogenetic protein (BMP), a platelet-derived growth factor (PDGF), vascular endothelial growth factor (VEGF), and/or placental growth factor (PIGF).

In some embodiments, an LbL film degrades, decomposes, and/or delaminates releasing growth factor. In some embodiments, growth factor is released at a rate of about 1 nanogram to about 20 nanograms growth factor per milligram of membrane per day. In some embodiments, growth factor is released over a period of at least about 2 days to at least about 30 days. In some embodiments, a multilayer LbL film is customized to control a rate of decomposition and/or delamination and release of growth factor.

In some embodiments, a multilayer LbL degrades, decomposes, and/or delaminates releasing a layer comprising PDGF and then degrades, decomposes, and/or delaminates releasing a layer comprising BMP. In some embodiments, a multilayer LbL degrades, decomposes, and/or delaminates releasing multiples layers of PDGF and then releasing multiple layers of BMP. In some embodiments, a multilayer LbL film that quickly degrades, decomposes, and/or delaminates quickly releasing PDGF, followed by a multilayer LbL film that slowly degrades, decomposes, and/or delaminates sustainably releasing of BMP.

In some embodiments, a multilayer LbL film is a tetralayer repeat unit of [Poly2/PAA/PDGF/PAA]. In some embodiments, a multilayer LbL film is a tetralayer repeat unit of [Poly2/PAA/BMP/PAA]. In some embodiments, a multilayer LbL film is a tetralayer repeat unit of [Poly2/PAA/BMP/PAA] comprising at least about 10 layers closest to a porous polymer membrane and a tetralayer repeat unit of [Poly2/PAA/PDGF/PAA] comprising at least about 10 layers subsequent to at least about 10 layers closest to a porous polymer membrane. In some embodiments, a multilayer LbL film is a tetralayer repeat unit of [Poly2/PAA/BMP/PAA] comprising about 40 layers closest to a porous polymer membrane and a tetralayer repeat unit of [Poly2/PAA/PDGF/PAA] comprising about 40 layers subsequent to about 40 layers closest to a porous polymer membrane.

### BRIEF DESCRIPTION OF THE DRAWINGS

The patent or application file contains at least one drawing executed in color. Copies of this patent or patent application publication with color drawings will be provided by the Office upon request and payment of the necessary fee.

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIGS. 1(a)-(f) Molecular structures of materials in the system. Hydrophobic PLGA is used to form the membrane. Poly2, PAA, BMP-2 and PDGF-BB are part of the bioactive interface that initiates the bone wound healing cascade. The bisphosphonate molecule alendronate is conjugated to PLGA.

FIG. 1(g) Schematic of the phase-inversion membrane formation process. (1) A PLGA-DMF solution is poured on a glass plate. (2) A doctor blade is used to spread the polymer solution uniformly on the glass plate and is (3) immersed into a deionized water bath. (4) The resulting film detaches from the glass substrate.

FIG. 1(h) Macroscopic image of the membrane structure that results in a uniform polymer support (scale bar, 8 mm).

FIG. 1(i) Scanning electron micrographs demonstrating a highly ordered cross section (scale bar, 10 μm).

FIG. 1(j) PLGA membrane coated with [Poly2/PAA/rhBMP-2/PAA]₄₀+[Poly2/PAA/rhPDGF-BB/PAA]₄₀ (scale bar, 2 microns).

FIG. 1(k) is a bar graph illustrating pore size distribution for the top surface of an example membrane (the surface away from the glass plate).

FIG. 1(l) is a bar graph illustrating pore size distribution for the bottom surface or an example membrane (the surface in contact with the glass plate).

FIGS. 2(a)-(b) Degradation profiles of the PLGA membrane in a rat calvaria as a function of the PLA:PGA ratio. Degradation was measured by dry mass difference and change in diameter. Data represent the means±s.e.m., n=4 per group per time point.

FIG. 2(c) is an illustration of a concentration gradient of growth factors administered to an animal by an embodiment of the present invention over time.

FIG. 2(d) is a plot of Radiant Efficiency measured versus Time (in days) of growth factors administered to an animal by an embodiment of the present invention.

FIG. 2(e) is a plot of single growth factor growth over time.

FIG. 2(f) is a plot of dual growth factor growth over time.

FIG. 3(a) Representative radiographs of bone formation around drilled implants with different films at 1, 2, and 4 weeks. Red broken circle indicates the location of the defect in each radiograph and has an 8 millimeters diameter. Defect closure was achieved in all animal groups with different treatment conditions within 4 weeks. n=5 per group.

FIGS. 3(b)-(c) The images in (a) were used to quantify bone volume and bone mineral density at 2 weeks (FIGS. 3(b) and 3(d)) and 4 weeks (FIGS. 3(c) and 3(e)) within the regions of interest marked by dotted red circles. Each point represents individual animal. Data are means±s.e.m. (n=5-6 per group). *p<0.05, **p<0.01, ***p<0.001, ns=not significant, ANOVA with Tukey post hoc test. All groups are compared with the mechanical properties of the M+B_{0.2}+P_{0.2} group.

FIG. 4(a) Each image is a cross section of the calvarial defect after 4 weeks, at which time different levels of bone tissue morphogenesis was observed at the defect site. The broken lines indicate the position of the defect site and are 8 millimeters apart. Collagen is represented by blue and osteocytes (mature bone) is represented by red. Sections were stained with Masson's trichrome stain and viewed under bright field microscopy.

FIGS. 4(b)-(d) Granulation tissue layer at 1, 2 and 4 weeks during bone repair in the M+B_{0.2}+P_{0.2} treatment group. The tissue gradually reduces in thickness from 1 to 4 weeks as bone repair is completed. Pieces of the PLGA membrane were observed in some section (scale bar, 30 microns). Arrows: red, PLGA membrane; yellow, granulation tissue layer.

FIG. 5(a) Stiffness from different groups are presented at 4 weeks after implantation. Data are means±s.e.m. (n=5 implants per group). *p<0.05; **p<0.01; ***p<0.001, ns=not significant, ANOVA with a Tukey post hoc test. All groups are compared with the mechanical properties of the M+B_{0.2}+P_{0.2} group.

FIG. 5(b) Failure load from different groups are presented at 4 weeks after implantation. Data are means±s.e.m. (n=5 implants per group). *p<0.05; **p<0.01; ***p<0.001, ns=not significant, ANOVA with a Tukey post hoc test. All groups are compared with the mechanical properties of the M+B_{0.2}+P_{0.2} group.

FIG. 6(a) Scanning electron micrographs of the membrane surface, top surface (scale bar, 1 micron).

FIG. 6(b) Scanning electron micrographs of the membrane surface, bottom surface (scale bar, 100 microns).

FIG. 7(a) Conjugation scheme of PLGA with alendronate.

FIG. 7(b) 31P-NMR of conjugated PLGA-alendronate product which shows the P signal at 18.7 parts per million relative to phosphoric acid standard corresponding to alendronate phosphonate moiety.

### DETAILED DESCRIPTION OF THE INVENTION

A description of example embodiments of the invention follows.

### DEFINITIONS

In order for the present disclosure to be more readily understood, certain terms are first defined below. Additional definitions for the following terms and other terms are set forth throughout the specification.

In this application, unless otherwise clear from context, the term “a” may be understood to mean “at least one.” As used in this application, the term “or” may be understood to mean “and/or.” In this application, the terms “comprising” and “including” may be understood to encompass itemized components or steps whether presented by themselves or together with one or more additional components or steps. Unless otherwise stated, the terms “about” and “approximately” may be understood to permit standard variation as would be understood by those of ordinary skill in the art. Where ranges are provided herein, the endpoints are included. As used in this application, the term “comprise” and variations of the term, such as “comprising” and “comprises,” are not intended to exclude other additives, components, integers or steps.

As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Administration”: As used herein, the term “administration” refers to the administration of a composition to a subject. Administration may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal and vitreal.

“Animal”: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, and/or worms. In some embodiments, an animal may be a transgenic animal, genetically-engineered animal, and/or a clone.

“Associated”: As used herein, the term “associated” typically refers to two or more entities in physical proximity with one another, either directly or indirectly (e.g., via one or more additional entities that serve as a linking agent), to form a structure that is sufficiently stable so that the entities remain in physical proximity under relevant conditions. In some embodiments, associated entities are covalently linked to one another. In some embodiments, associated entities are non-covalently linked. In some embodiments, associated entities are linked to one another by specific non-covalent interactions (i.e., by interactions between interacting ligands that discriminate between their interaction partner and other entities present in the context of use, such as, for example. streptavidin/avidin interactions, antibody/antigen interactions, etc.). Alternatively or additionally, a sufficient number of weaker non-covalent interactions can provide sufficient stability for moieties to remain associated. Exemplary non-covalent interactions include, but are not limited to, affinity interactions, metal coordination, physical adsorption, host-guest interactions, hydrophobic interactions, pi stacking interactions, hydrogen bonding interactions, van der Waals interactions, magnetic interactions, electrostatic interactions, dipole-dipole interactions, etc.

“Biocompatible:” As used herein, the term “biocompatible” is intended to describe any material which does not elicit a substantial detrimental response in vivo.

“Biodegradable”: As used herein, the term “biodegradable” refers to materials that, when introduced into cells, are broken down (e.g., by cellular machinery, such as by enzymatic degradation, by hydrolysis, and/or by combinations thereof) into components that cells can either reuse or dispose of without significant toxic effects on the cells. In certain embodiments, components generated by breakdown of a biodegradable material are biocompatible and therefore do not induce significant inflammation and/or other adverse effects in vivo. In some embodiments, biodegradable polymer materials break down into their component monomers. In some embodiments, breakdown of biodegradable materials (including, for example, biodegradable polymer materials) involves hydrolysis of ester bonds. Alternatively or additionally, in some embodiments, breakdown of biodegradable materials (including, for example, biodegradable polymer materials) involves cleavage of urethane linkages. Exemplary biodegradable polymers include, for example, polymers of hydroxy acids such as lactic acid and glycolic acid, including but not limited to poly(hydroxyl acids), poly(lactic acid)(PLA), poly(glycolic acid)(PGA), poly(lactic-co-glycolic acid)(PLGA), and copolymers with PEG, polyanhydrides, poly(ortho)esters, polyesters, polyurethanes, poly(butyric acid), poly(valeric acid), poly(caprolactone), poly(hydroxyalkanoates, poly(lactide-co-caprolactone), blends and copolymers thereof. Many naturally occurring polymers are also biodegradable, including, for example, proteins such as albumin, collagen, gelatin and prolamines, for example, zein, and polysaccharides such as alginate, cellulose derivatives and polyhydroxyalkanoates, for example, polyhydroxybutyrate blends and copolymers thereof. Those of ordinary skill in the art will appreciate or be able to determine when such polymers are biocompatible and/or biodegradable derivatives thereof (e.g., related to a parent polymer by substantially identical structure that differs only in substitution or addition of particular chemical groups as is known in the art).

“Burst-free release”: The term “burst-free release” is used herein to distinguish from “burst-release” which, as is known in the art, refers to release of an agent from a composition with a release profile characterized by a burst in which a significant amount of the agent is released in a relatively short amount of time. Often, such a burst occurs early in a release profile. In some embodiments, a burst is significantly higher than otherwise seen within the release profile. In some embodiments, a burst release is an unsustained release. In some embodiments, a burst-free release is characterized by the absence of a single significant release burst. In some embodiments, a burst-free release is characterized in that the degree of variation in release rate over time does not fluctuate beyond acceptable values understood in the art (e.g., a therapeutic window of a particular agent). In some embodiments, burst-free release is characterized by the absence of any single burst in which more than 20% of the agent is released within a time period that is less than 10% of the total time required to substantially release all of the material. In some embodiments, a burst-free release is characterized by releasing less than about 10%, less than about 20%, less than about 30%, less than about 40%, or less than about 50% of an agent for delivery in the first 1, 2, 5, 10, 12 or 24 hours of releasing.

“Comparable”: The term “comparable”, as used herein, refers to two or more agents, entities, situations, sets of conditions, etc. that may not be identical to one another but that are sufficiently similar to permit comparison therebetween so that conclusions may reasonably be drawn based on differences or similarities observed. Those of ordinary skill in the art will understand, in context, what degree of identity is required in any given circumstance for two or more such agents, entities, situations, sets of conditions, etc. to be considered comparable.

“Conjugated”: As used herein, the terms “conjugated,” “linked,” “attached,” and “associated with,” when used with respect to two or more moieties, means that the moieties are physically associated or connected with one another, either directly or via one or more additional moieties that serves as a linking agent, to form a structure that is sufficiently stable so that the moieties remain physically associated under the conditions in which structure is used. Typically the moieties are attached either by one or more covalent bonds or by a mechanism that involves specific binding. Alternately, a sufficient number of weaker interactions can provide sufficient stability for moieties to remain physically associated.

“Dosage form”: As used herein, the term “dosage form” refers to a physically discrete unit of a therapeutic agent for administration to a subject. Each unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen).

“Hydrolytically degradable”: As used herein, the term “hydrolytically degradable” is used to refer to materials that degrade by hydrolytic cleavage. In some embodiments, hydrolytically degradable materials degrade in water. In some embodiments, hydrolytically degradable materials degrade in water in the absence of any other agents or materials. In some embodiments, hydrolytically degradable materials degrade completely by hydrolytic cleavage, e.g., in water. By contrast, the term “non-hydrolytically degradable” typically refers to materials that do not fully degrade by hydrolytic cleavage and/or in the presence of water (e.g., in the sole presence of water).

“Hydrophilic”: As used herein, the term “hydrophilic” and/or “polar” refers to a tendency to mix with, or dissolve easily in, water.

“Hydrophobic”: As used herein, the term “hydrophobic” and/or “non-polar”, refers to a tendency to repel, not combine with, or an inability to dissolve easily in, water.

“Physiological conditions”: The phrase “physiological conditions”, as used herein, relates to the range of chemical (e.g., pH, ionic strength) and biochemical (e.g., enzyme concentrations) conditions likely to be encountered in the intracellular and extracellular fluids of tissues. For most tissues, the physiological pH ranges from about 6.8 to about 8.0 and a temperature range of about 20-40 degrees Celsius, about 25-40 degrees Celsius, about 30-40 degrees Celsius, about 35-40 degrees Celsius, about 37 degrees Celsius, atmospheric pressure of about 1. In some embodiments, physiological conditions utilize or include an aqueous environment (e.g., water, saline, Ringers solution, or other buffered solution); in some such embodiments, the aqueous environment is or comprises a phosphate buffered solution (e.g., phosphate-buffered saline).

“Polyelectrolyte”: The term “polyelectrolyte”, as used herein, refers to a polymer which under a particular set of conditions (e.g., physiological conditions) has a net positive or negative charge. In some embodiments, a polyelectrolyte is or comprises a polycation; in some embodiments, a polyelectrolyte is or comprises a polyanion. Polycations have a net positive charge and polyanions have a net negative charge. The net charge of a given polyelectrolyte may depend on the surrounding chemical conditions, e.g., on the pH.

“Polypeptide”: The term “polypeptide” as used herein, refers to a string of at least three amino acids linked together by peptide bonds. In some embodiments, a polypeptide comprises naturally-occurring amino acids; alternatively or additionally, in some embodiments, a polypeptide comprises one or more non-natural amino acids (i.e., compounds that do not occur in nature but that can be incorporated into a polypeptide chain; see, for example, http://www.cco.caltech.edu/^{˜}dadgrp/Unnatstruct.gif, which displays structures of non-natural amino acids that have been successfully incorporated into functional ion channels) and/or amino acid analogs as are known in the art may alternatively be employed). For example, a polypeptide can be a protein. In some embodiments, one or more of the amino acids in a polypeptide may be modified, for example, by the addition of a chemical entity such as a carbohydrate group, a phosphate group, a farnesyl group, an isofarnesyl group, a fatty acid group, a linker for conjugation, functionalization, or other modification, etc.

“Polysaccharide”: The term “polysaccharide” refers to a polymer of sugars. Typically, a polysaccharide comprises at least three sugars. In some embodiments, a polypeptide comprises natural sugars (e.g., glucose, fructose, galactose, mannose, arabinose, ribose, and xylose); alternatively or additionally, in some embodiments, a polypeptide comprises one or more non-natural amino acids (e.g, modified sugars such as 2′-fluororibose, 2′-deoxyribose, and hexose).

“Small molecule”: As used herein, the term “small molecule” is used to refer to molecules, whether naturally-occurring or artificially created (e.g., via chemical synthesis), having a relatively low molecular weight and being an organic and/or inorganic compound. Typically, a “small molecule” is monomeric and have a molecular weight of less than about 1500 g/mol. In general, a “small molecule” is a molecule that is less than about 5 kilodaltons in size. In some embodiments, a small molecule is less than about 4 kilodaltons, 3 kilodaltons, about 2 kilodaltons, or about 1 kilodalton. In some embodiments, the small molecule is less than about 800 daltons, about 600 daltons, about 500 daltons, about 400 daltons, about 300 daltons, about 200 daltons, or about 100 daltons. In some embodiments, a small molecule is less than about 2000 grams/mol, less than about 1500 grams/mol, less than about 1000 grams/mol, less than about 800 grams/mol, or less than about 500 grams/mol. In some embodiments, a small molecule is not a polymer. In some embodiments, a small molecule does not include a polymeric moiety. In some embodiments, a small molecule is not a protein or polypeptide (e.g., is not an oligopeptide or peptide). In some embodiments, a small molecule is not a polynucleotide (e.g., is not an oligonucleotide). In some embodiments, a small molecule is not a polysaccharide. In some embodiments, a small molecule does not comprise a polysaccharide (e.g., is not a glycoprotein, proteoglycan, glycolipid, etc.). In some embodiments, a small molecule is not a lipid. In some embodiments, a small molecule is a modulating agent. In some embodiments, a small molecule is biologically active. In some embodiments, a small molecule is detectable (e.g., comprises at least one detectable moiety). In some embodiments, a small molecule is a therapeutic. Preferred small molecules are biologically active in that they produce a local or systemic effect in animals, preferably mammals, more preferably humans. In certain preferred embodiments, the small molecule is a drug. Preferably, though not necessarily, the drug is one that has already been deemed safe and effective for use by the appropriate governmental agency or body. For example, drugs for human use listed by the FDA under 21 C.F.R. §§330.5, 331 through 361, and 440 through 460; drugs for veterinary use listed by the FDA under 21 C.F.R. §§500 through 589, incorporated herein by reference, are all considered acceptable for use in accordance with the present application.

“Stable”: The term “stable,” when applied to compositions herein, means that the compositions maintain one or more aspects of their physical structure and/or activity over a period of time under a designated set of conditions. In some embodiments, the period of time is at least about one hour; in some embodiments, the period of time is about 5 hours, about 10 hours, about one (1) day, about one (1) week, about two (2) weeks, about one (1) month, about two (2) months, about three (3) months, about four (4) months, about five (5) months, about six (6) months, about eight (8) months, about ten (10) months, about twelve (12) months, about twenty-four (24) months, about thirty-six (36) months, or longer. In some embodiments, the period of time is within the range of about one (1) day to about twenty-four (24) months, about two (2) weeks to about twelve (12) months, about two (2) months to about five (5) months, etc. In some embodiments, the designated conditions are ambient conditions (e.g., at room temperature and ambient pressure). In some embodiments, the designated conditions are physiologic conditions (e.g., in vivo or at about 37 degrees Celsius for example in serum or in phosphate buffered saline). In some embodiments, the designated conditions are under cold storage (e.g., at or below about 4 degrees Celsius, −20 degrees Celsius, or −70 degrees Celsius). In some embodiments, the designated conditions are in the dark.

“Substantially”: As used herein, the term “substantially”, and grammatic equivalents, refer to the qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result.

“Sustained release”: The term “sustained release” is used herein in accordance with its art-understood meaning of release that occurs over an extended period of time. The extended period of time can be at least about 3 days, about 5 days, about 7 days, about 10 days, about 15 days, about 30 days, about 1 month, about 2 months, about 3 months, about 6 months, or even about 1 year. In some embodiments, sustained release is substantially burst-free. In some embodiments, sustained release involves steady release over the extended period of time, so that the rate of release does not vary over the extended period of time more than about 5%, about 10%, about 15%, about 20%, about 30%, about 40% or about 50%. In some embodiments, sustained release involves release with first-order kinetics. In some embodiments, sustained release involves an initial burst, followed by a period of steady release. In some embodiments, sustained release does not involve an initial burst. In some embodiments, sustained release is substantially burst-free release.

“Treating”: As used herein, the term “treating” refers to partially or completely alleviating, ameliorating, relieving, inhibiting, preventing (for at least a period of time), delaying onset of, reducing severity of, reducing frequency of and/or reducing incidence of one or more symptoms or features of a particular disease, disorder, and/or condition. In some embodiments, treatment may be administered to a subject who does not exhibit symptoms, signs, or characteristics of a disease and/or exhibits only early symptoms, signs, and/or characteristics of the disease, for example for the purpose of decreasing the risk of developing pathology associated with the disease. In some embodiments, treatment may be administered after development of one or more symptoms, signs, and/or characteristics of the disease.

Various embodiments of composite devices and methods according to the present invention are described in detail herein. In particular, composite devices and methods for assembling composite devices are disclosed. Provided composite devices when assembled and placed in contact with a bone or tissue defect are useful for isolating a defect site and providing targeted delivery of growth factors to a defect.

In some embodiments, composite devices of the present invention when placed on a defect site cover or enclose a defect. In some embodiments, composites devices of the present invention restrict fibrous tissue ingrowth that may result in a disease, disorder or condition at a defect site, for example, fibrous dysplasia.

In some embodiments, composite devices of the present invention provide delivery of growth factor to a targeted area, thereby minimizing unwanted biological effects and/or toxicity.

In some embodiments, composites devices of the present invention are characterized by, for example, controlled and/or sustained delivery of growth factors. In some embodiments, growth factor enhances bone repair through stimulating cell growth. In some embodiments, composites devices of the present invention support cell proliferation. In some embodiments, composite devices of the present invention induce rapid repair of a large bone and/or tissue defects.

In some embodiments, composite devices of the present invention are biodegradable. In some embodiments, composite devices of the present invention are characterized in that such devices are resorbable. In some embodiments, new tissue or bone identical to original tissue eventually replaces composite devices of the present invention.

When compared with composites devices of the present invention, conventional treatment options for repair of large bone and/or tissue defects are susceptible to failure due to the low rate of defect closure or the difficulty and/or complexity of the associated surgical procedures.

Bone and bone tissue will self-regenerate when the damage or defect is small, such as a crack or a minor fracture. In animals, the body will respond to a fracture or defect quickly causing stabilization at the site of the defect. Repair and remodeling can occur when a fracture gap is small enough so that the gap may be bridged. But, large bone injuries do not spontaneously close. The healing process in an animal model has been well characterized and an 8 mm defect size is too large to heal without intervention. Bone must be stabilized when the defects are large enough that self-regeneration is not possible. Moreover, in the absence of localized morphogenetic cellular stimuli, multicellular processes necessary for bone tissue formation cannot be easily induced.

Traditional options for large bone defect repair have included grafting real bone at a defect site or grafting using a synthetic bone material at a defect site. Grafting materials have been extensively studied for their potential role in regenerating bone tissue and restoring functional properties. However, the primary treatment and closure of large-area bone defects continues to face major technical challenges.

The gold standard for treatment and closure of large-area bone defects for craniomaxillofacial (CMF) reconstruction, segmental bone defects, and spine fusion is currently autograft transplantation. Grafting via an autograft transplantation uses real bone, from a patient's body. Autologous bone grafts promote bone healing in fractures and provide structural support during healing and reconstructive surgery. Osteoinduction, stimulating new bone; osteoconduction, providing a support scaffold; and osteogenesis, furthering new bone growth is highest in autograft transplantation when compared with other graft materials, such as allografts (cadaver), xenografts (non-human animal), or synthetic grafts. Autologous bone typically both incorporates into a defect site with greater predictability and without an unfavorable immunogenic response associated with other grafts.

While autograft transplantation is the benchmark treatment for large bone defects, related complications reduce its effectiveness. Donor bone for an autograft transplantation is typically harvested from the iliac crest and is in limited supply. Moreover, harvesting of an autograft may result in disease, disorder, or condition, for example, sever herniation, vascular injury, donor site infection, neurologic injuries, hematoma, iliac fracture, and/or morbidity. Harvesting of bone grafts can be associated with morbidity at the donor site. Morbidity includes damage to nerves or primary blood vessels that are adjacent to a defect site. Additionally, as indicated above bone grafting surgeries are complex and pose a significant health risk to the patient so that patients frequently require revision surgery because a graft had failed to properly heal. CMF reconstruction is particularly challenging due to the complexity of reconstructing a three dimensional facial geometry with fidelity while protecting the underlying delicate organ systems. Again, multiple surgeries are often necessary. The result is a complex permanent implant system that can lead to permanent deformities, functional impairment, and an alteration of physical appearance.

Grafting using synthetic materials is an alternative transplantation option. Synthetic materials, such as calcium phosphate, are widely used in dental implant procedures. Synthetic graft materials are made of a scaffold of inorganic materials with mechanical properties similar to those of real bone. But, because these synthetic materials are manufactured, they are not limited in supply as is real bone.

Synthetic bone graft transplantation however also has its limitations. Synthetic bone grafts likely will not integrate with the host bone of a patient because, for example, a mismatch in a lattice constant between host bone and they synthetic bone may exist thereby disrupting an interface between the materials. Calcium phosphate based synthetic scaffolds are rigid and brittle in nature, and may not easily conform to a defect. Additionally, differences in the mechanical properties between the synthetic bone and the host bone cause a distribution of load differentially transmitted through the synthetic graft when compared with host bone causing disease, disorder, or condition of existing host bone, for example, osteoporosis. Synthetic bone is typically not resorbable compounding these issues. Moreover, regulating a synthetic implant once installed in the body of a patient typically requires revision surgery and often multiple revision surgeries to correct issues that arise.

Many bone transplant patients with the most acute need are not viable candidates for an autograft, allograft or synthetic graft transplantation. The risk of disease, disorder or condition as shown above is prevalent. Additionally, patients in acute need often already have a fairly compromised bone structure or due to bone loss with age lack enough bone to properly implant a graft. As a result, the pain caused by a grafting procedure may be greater than the pain alleviated through a successful grafting procedure.

Recent attention in the field of tissue engineering for repair of large bone defects has been on developing permanent scaffolds and degradable scaffolds for facilitating tissue regeneration. Permanent scaffolds include, for example, metals, alloys, etc. However, metallic and alloy implants can induce a foreign body response throughout the patient's lifetime. Additionally, permanent polymer scaffolds bone lack tunable degradation behavior, which can often hinder bone regeneration and remodeling processes. Hydrogel-based delivery systems are a degradable alternative and can effectively present biologics, such as growth factors; however, these systems lack mechanical integrity and compression resistance necessary for large area bone reconstruction. In fact, the isotropic nature of these hydrogels may hinder complex CMF reconstruction procedures that require recapitulation of specific geometries and guided regeneration in situ.

Bone healing and regeneration are orchestrated via the action of a number of growth factors. In the context of bone tissue engineering, bone morphogenetic protein (BMP) and platelet derived growth factor (PDGF) are two of the most prominent growth factors for the treatment of defects in bone presenting as orthopedic and oral and maxillofacial problems. Delivery for these regulatory molecules is essential for their effectiveness. Bolus release of these growth factors from injectable or implantable carriers and depots results in a rapid clearance of protein from a defect site by serum proteins and is counterproductive in repair. In fact, carriers containing BMP in large quantities have been used in the clinic for BMP release and clearance.

The inability to deliver growth factor or modulate growth factor dose for an extended period from a carrier has resulted in suboptimal tissue regeneration and undesired side effects in traditional devices. Delivery vehicles for growth factors have been unable to: a) cover large defects in such a way as to maintain a bony contour; b) provide a controlled tunable and sequential release of the growth factors; and c) enable a lower dose of the growth factor to be used without reducing the osteogenic effectiveness of the device.

### Composite Devices

In some embodiments, composite devices of the present invention are tailored to fit a defect site. In some embodiments, composite devices of the present invention are characterized in that they provide necessary stability to support a defect site during repair. In some embodiments, composite devices of the present invention are characterized by targeted delivery.

In some embodiments, composite devices of the present invention are characterized in that they degrade over a period to release growth factor and/or multiple growth factors that can induce and sustain bone regeneration. In some embodiments, simultaneously delivering multiple biological growth factors that mimic a natural healing cascade to a defect site will result in controlled bone formation in large defects. In some embodiments, controlled delivery of growth factor from a porous polymer membrane will (i) recapitulate cellular regenerative processes and substantially enhance bone formation by inducing angiogenesis followed by osteogenesis and, (ii) promote rapid bone repair and provide a supporting structure to guide the regenerative process where needed for repairing large CMF defects and overcoming limitations as described above. In some embodiments, composite devices and methods of the present invention promote bone matrix formation by endogenous progenitor cells, and provide biological cues to induce tissue bridging across the wound. In some embodiments, composite devices of the present invention are further characterized in that they can both induce bone healing and regenerate a new bone tissue matrix that restores functional properties as well as being integrated with the native bone and therefore withstand load bearing movement.

In some embodiments, composite devices of the present invention are further characterized in that they are biodegradable and/or resorbable and over time native tissue or bone displaces a composite device. In some embodiment, composite devices degrade, decompose, and/or delaminate when contacting bone and/or tissue in a physiological environment inducing aspects of a natural healing cascade.

### Porous Polymer Membranes

In some embodiments, a composite device comprising a porous polymer membrane was formed.

In some embodiments, composite devices comprising a porous polymer membrane provide structure to support bone regeneration. In some embodiments, composite devices comprising a porous polymer membrane provide to isolate a defect site during bone and/or tissue repair and regeneration.

In some embodiments, a porous polymer membrane is utilized to enclose, cover, fill, and/or isolate a defect. In some embodiments, a porous polymer membrane and/or a composite device is conformable, for example so that its shape can adapt to a defect. In some embodiments, a porous polymer membrane provides a scaffold to structurally support bone and tissue regeneration at and around a defect site. In some embodiments, a porous polymer membrane and/or a device may be sized, for example, by cutting or molding so that its shape fits a defect or is matched to a defect or a wound. Those skilled in the art will appreciate that, in some embodiments, a device and/or a membrane is not required to exactly conform to or match a shape of a defect site. In some embodiments, a device and/or a membrane is not exactly sized to match a defect site. In some embodiments, a device and/or a membrane is larger or smaller than a defect site. In some embodiments, a device and/or a membrane overlaps a defect or a wound. In some embodiments, at least a portion of a defect remains uncovered or unfilled by a device and/or a membrane.

In some embodiments, a porous polymer membrane is associated with growth factor. In some embodiments, a porous polymer membrane is not associated with growth factor.

In some embodiments, a porous polymer membrane provides a scaffold to support delivery of growth factor. In some embodiments, a porous polymer membrane degrades decomposes, and/or delaminates on contact with an environment under physiological conditions. In some embodiments, a porous polymer membrane degrades, decomposes, and/or delaminates releasing growth factor. In some embodiments, a device is characterized in that at least one growth factor releases having a staggered release. In some embodiments, a device is characterized in that at least one growth factor releases having a concurrent release. In some embodiments, a staggered release is characterized in that a device is designed or arranged for alternating release, wherein multiple growth factors are alternately released and/or growth factor is alternately released from a device and/or membrane. In some embodiments, a concurrent release is characterized in that a device is designed or arranged for substantially simultaneous release, wherein multiple growth factors are substantially simultaneous released and/or growth factor is substantially simultaneous released from a device and/or membrane.

In some embodiments, a rate of membrane degradation, decomposition, and/or delamination is critical to bone healing. In some embodiments, different tissue types repair at different rates, for example, bone repair may be induced within about two to three weeks. In some embodiments, release of growth factor from a composite device is controllable by a porous polymer membrane. In some embodiments, a rate of release of growth factor is controllable through degradation of a porous polymer membrane. In some embodiments, membrane activity and degradation, decomposition, and/or delamination rate are varied by adjustments in membrane parameters, for example, polymer identity, thickness, pore size, porosity, variation in porosity across the membrane, and an addition of functional groups.

In some embodiments a porous polymer membrane is biodegradable. In some embodiments, a porous polymer membrane comprises any biodegradable polymer. In some embodiments, a polymer is natural or synthetic. In some embodiments, degradable polymers known in the art include, for example, certain polyesters, polyanhydrides, polycaptolactone, polyorthoesters, polyphosphazenes, polyphosphoesters, certain polyhydroxyacids, polypropylfumerates, polycaprolactones, polyamides, poly(amino acids), polyacetals, polyethers, biodegradable polycyanoacrylates, biodegradable polyurethanes, poly(glycerol sebacates), elastomeric poly(glycerol sebacates polysaccharides), polypyrrole, polyanilines, polythiophene, polystyrene, polyesters, polyureas, poly(ethylene vinyl acetate), polypropylene, polymethacrylate, polyethylene, polycarbonates, poly(ethylene oxide), polysaccharides, copolymers, and combinations thereof. For example, specific biodegradable polymers that may be used include but are not limited to polylysine (e.g., poly(L-lysine) (“PLL”)), poly(lactic acid) (“PLA”), poly(glycolic acid) (“PGA”), polylactic acid/poly(glycolide-colactide) copolymer (“PLGA”), poly(caprolactone) (“PCL”), poly(lactide-co-glycolide) (“PLG”), poly(lactide-co-caprolactone) (“PLC”), poly(glycolide-co-caprolactone) (“PGC”), poly(styrene sulfonate) (“SPS”), poly(acrylic acid) (“PAA”), linear poly(ethylene imine) (“LPEI”), poly(diallyldimethyl ammonium chloride) (“PDAC”), and poly(allylamine hydrochloride) (“PAH”). Another exemplary degradable polymer is poly(beta-amino esters), which may be suitable for use in accordance with the present application. Those skilled in the art will recognize that this is an exemplary, not comprehensive, list of polymers.

In some embodiments, a porous polymer membrane is comprised of polycaprolactone (PCL).

In some embodiments, a PCL membrane degrades, decomposes, and/or delaminates with a degradation half-life of up to one year for cranial defect healing.

In some embodiments, a porous polymer membrane comprises polylactic acid/poly(glycolide-colactide) copolymer (“PLGA”). In some embodiments, an aqueous polymer solution, a water immersion, precipitates PLGA to form a membrane.

In some embodiments, in vivo mechanical and degradation properties of a porous polymer membrane are a function of a ratio of PLA to PGA in a polymer backbone. In some embodiments, a lower ratio results in more elastic membranes. In some embodiments, more elastic membranes degrade faster. In some embodiments, a ratio that yields a degradation half-life of about four weeks coincides with bone growth. In some embodiments, a PLA:PGA ratio of 50:50 yielded a desirable degradation profile for cranial defect healing with a degradation of half-life of about four weeks.

In some embodiments, different porous polymer membranes degrade, decompose, and/or delaminate over different periods according to thicknesses. In some embodiments, a porous polymer membrane thickness varies from about 50 microns to about 200 microns. In some embodiments, a porous polymer membrane thickness varies from about 100 microns to about 150 microns. In some embodiments, a porous polymer membrane thickness varies from about 110 microns to about 130 microns. In some embodiments, a porous polymer membrane thickness is about 110 microns. In some embodiments, a porous polymer membrane thickness is about 115 microns. In some embodiments, a porous polymer membrane thickness is about 120 microns. In some embodiments, a porous polymer membrane thickness is about 125 microns. In some embodiments, a porous polymer membrane thickness is about 130 microns. In some embodiments, the thickness (dry) of the membrane was 120±10 microns, measured by a micrometer.

In some embodiments, a poly(lactic-co-glycolic) acid (PLGA) porous polymer membrane supports active cell proliferation in vascular and bone tissue. In some embodiments, a PLGA porous polymer membrane with interconnected pores allows for association and sequestration of active biologics to induce and promote bone and tissue regeneration. In some embodiments, a composite device comprising a porous polymer membrane further allows direct control of a bone regenerative process to rapidly induce repair in critical size defect with mechanically competent bone.

In some embodiments, a porosity of a membrane is varied by doping a polymer solution with biocompatible surfactants. In some embodiments, a polymer solutions is adding dopants such as poly(ethylene oxide) (PEO) and polyvinylpyrrolidone (PVP) varies a porosity of a porous polymer membrane.

In some embodiments, pore size varies within a cross section of a porous polymer membrane between a top surface of a porous polymer membrane and a bottom surface of a porous polymer membrane. In some embodiments, a distribution of a pore size increases in a cross section of a porous polymer membrane between a top surface of a porous polymer membrane and a bottom surface of a porous polymer membrane. In some embodiments, a pore size varies from about 2 microns on a top surface to 200 microns on a bottom surface of a porous polymer membrane. In some embodiments, a pore size varies from about 10 microns on a top surface to 190 microns on a bottom surface of a porous polymer membrane. In some embodiments, a pore size varies from about 25 microns on a top surface to 150 microns on a bottom surface of a porous polymer membrane. In some embodiments, a pore size varies from about 50 microns on a top surface to 100 microns on a bottom surface of a porous polymer membrane.

In some embodiments, a porous polymer membrane with a distribution of pore size that increases in a cross section of a porous polymer membrane between a top surface of a porous polymer membrane and a bottom surface of a porous polymer membrane results in a bifunctional membrane. In some embodiments, a bifunctional membrane is permeable on top and impermeable on bottom. In some embodiments, a bifunctional membrane is impermeable on top and permeable on bottom. In some embodiments, bifunctionality supports a top surface that releases drugs and a bottom surface for cell migration. In some embodiments, a variation is porosity allows for functionalization of and permits an addition of growth factor to a porous polymer membrane. In some embodiments, a top layer thickness may be tuned to permit incorporation of drugs. In some embodiments, a top surface can release drugs and/or growth factor to support bone formation.

In some embodiments a hydrophobic agent associates with a top surface of a porous polymer membrane. In some embodiments a bisphosphonate is a hydrophobic agent. In some embodiments, a bisphosphonate conjugates to a porous polymer membrane. In some embodiments, a bisphosphonate binds to bone and encourages rapid bone deposition. In some embodiments, a bisphosphonate is an alendronate.

In some embodiments, a PLGA porous polymer membrane has end-groups conjugated with alendronate. In some embodiments, a PLGA terminal hydroxyl group is activated with p-nitrophenyl chloroformate to generate a highly efficient chloroformate leaving group at a PLGA chain end. In some embodiments, a highly efficient chloroformate leaving group at a PLGA chain end is quantitatively substituted by an alendronate amine group. A reaction placing a negatively charged phosphonate end group at an end of the hydrophobic PLGA backbone, generates an amphiphilic molecule. In some embodiments, an alendronate moiety extends towards the hydrophilic environment, making the alendronate accessible on the surface of the membrane generating an end-modified PLGA.

In some embodiments, alendronate enhances bone formation by modulating bone resorption by binding to osseous tissue and inhibiting osteoclast resorption of bone. In some embodiments, alendronate is combined with nanoparticles that act as drug carriers targeting bone tissue. In some embodiments, a PLGA porous polymer membrane with end-groups conjugated with alendronate, displays a high affinity for hydroxyapatite and is used to assist in clinical management of osteoporosis. In some embodiments, alendronate groups bind to hydroxyapatite, inhibiting bone resorption and potentially leading to rapid bone formation. In some embodiments, end-functionalization of alendronate to a PLGA porous polymer membrane having a 50:50 ratio of PLA:PGA in a polymer backbone did not noticeably alter porous polymer membrane in vivo degradation kinetics.

In some embodiments, a porous polymer membrane comprises an agent to be delivered. In some embodiments, a porous polymer membrane comprises a small molecule. In some embodiments, a porous polymer membrane degrades releasing a small molecule. In some embodiments, a small molecule is gentamicin. In some embodiments, a small molecule is associated with a porous polymer membrane. In some embodiments, a small molecule releases as a porous polymer membrane degrades.

In another embodiment, a porous polymer membrane comprises a first agent and a second agent to be delivered, the first and second agents to be delivered being released as the porous polymer membrane degrades, decomposes and/or delaminates. One or both agents are associated or covalently attached (conjugated) to the porous polymer membrane. In another embodiment, the first and second agents to be delivered are small molecules. In another embodiment, the first small molecule is an antibiotic. In another embodiment, the antibiotic is gentamicin. In another embodiment the second small molecule is an anti-inflammatory agent. In another embodiment, the anti-inflammatory agent is ibuprofen. In another embodiment, the porous polymer membrane is PLGA, the antibiotic is gentamicin, the anti-inflammatory is ibuprofen, and the PLGA-gentamicin conjugate is represented by the structure below:

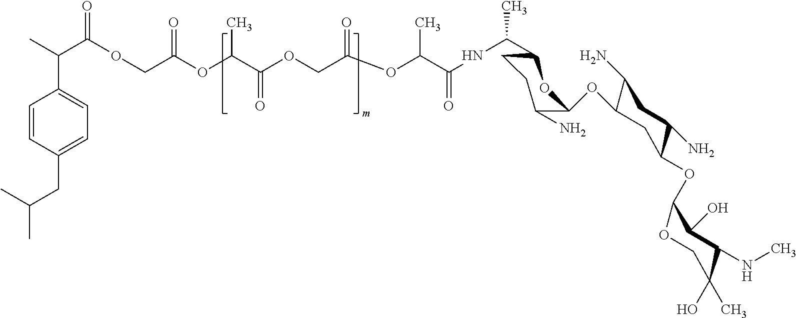

wherein m is an integer.

### Biodegradable LbL Films

In some embodiments, composite devices of the present invention also include a degradable multilayer film. In some embodiments, a porous polymer membrane provides a surface for coating with a multilayer film. In some embodiments, a porous polymer membrane is coated with a multilayer film. In some embodiments, a porous polymer membrane is partially coated with a multilayer film. In some embodiments, a multilayer film is coated on a porous polymer membrane.

In some embodiments, composite devices also include a multilayer film associated with growth factor. In some embodiments, a natural healing cascade may be created when multilayer films associated with growth factor are designed and/or tailored to release growth factor. In some embodiments, composite devices include a multilayer film associated with multiple growth factors. In some embodiments, provided composite devices including a degradable multilayer film are characterized by, for example, high loading, substantially burst-free release, sustained release, and/or effective release of growth factor. In some embodiments, critical size defects heal when composite devices comprising a porous polymer membrane conformally coated with growth factor using a Layer-by-Layer (LbL) approach are implanted at a defect site.

In some embodiments, composite devices comprise a multilayer film coated on a surface of a porous polymer membrane. In some embodiments, a multilayer film is coated on a porous polymer membrane using a polyelectrolyte multilayer (PEM) film. In some embodiments, a PEM is an LbL film, which are nanostructured films formed by an LbL technique of iterative adsorption of alternately charged materials coated a porous polymer membrane surface. In some embodiments, PEMs can sequester and elute multiple growth factors in a controlled, pre-programmed manner over several weeks or months; release profiles can be easily tuned by modifying a multilayer LbL film architecture.

In some embodiments, a device includes a porous polymer membrane with an LbL film associated with a porous polymer membrane each comprising at least one growth factor. In some embodiments, a device includes an LbL film comprising growth factor associated with a porous polymer membrane without growth factor.

In some embodiments, a multilayer LbL degrades, decomposes, and/or delaminates on contact with an environment under physiological conditions. In some embodiments, a multilayer LbL sequentially releases multiple growth factor. In some embodiments, a device is characterized in that at least one growth factor releases having a staggered release. In some embodiments, a device is characterized in that at least one growth factor releases having a concurrent release. In some embodiments, a staggered release is characterized in that a device is designed or arranged for alternating release, wherein multiple growth factors are alternately released and/or growth factor is alternately released from a device, membrane, and/or film. In some embodiments, a concurrent release is characterized in that a device is designed or arranged for substantially simultaneous release, wherein multiple growth factors are substantially simultaneous released and/or growth factor is substantially simultaneous released from a device, membrane, and/or film.

In some embodiments, a multilayer LbL degrades, decomposes, and/or delaminates releasing a layer comprising a first growth factor and then degrades, decomposes, and/or delaminates releasing a layer comprising a second growth factor. In some embodiments, a multilayer LbL degrades, decomposes, and/or delaminates releasing a layer comprising a first growth factor and then degrades, decomposes, and/or delaminates releasing a layer comprising a second growth factor and then degrades, decomposes, and/or delaminates releasing a layer comprising a third growth factor. In some embodiments, a sequential release of growth factor includes releasing a fourth growth factor, a fifth growth factor, a sixth growth factor, and so on. In some embodiments, multiple layers of a multilayer LbL comprising a first growth factor degrades, decomposes, and/or delaminates releasing a first growth factor followed by multiple layers of a multilayer LbL comprising a second growth factor degrades, decomposes, and/or delaminates releasing a first growth factor and then releasing multiple layers of BMP. In some embodiments, a multilayer LbL film that quickly degrades, decomposes, and/or delaminates quickly releasing PDGF, followed by a multilayer LbL film that slowly degrades, decomposes, and/or delaminates sustainably releasing of BMP. In some embodiments, a multilayer LbL concurrently releases multiple growth factor.

Multilayer films described herein can be made of or include one or more LbL films. LbL films may have any of a variety of film architectures (e.g., numbers of layers, thickness of individual layers, identity of materials within films, nature of surface chemistry, presence and/or degree of incorporated materials, etc.), as appropriate to the design and application of coated substrates as described herein.

In many embodiments, LbL films are comprised of multilayer units; each unit comprising individual layers. In some embodiments, adjacent layers are associated with one another via non-covalent interactions. Exemplary non-covalent interactions include, but are not limited to ionic interactions, hydrogen bonding interactions, affinity interactions, metal coordination, physical adsorption, host-guest interactions, hydrophobic interactions, pi stacking interactions, van der Waals interactions, magnetic interactions, dipole-dipole interactions and combinations thereof.

LbL films may be comprised of multilayer units in which alternating layers have opposite charges, such as alternating anionic and cationic layers. Alternatively or additionally, LbL films for use in accordance with the present invention may be comprised of (or include one or more) multilayer units in which adjacent layers are associated via non-electrostatic interactions.

According to the present disclosure, an LbL film may be comprised of one or more multilayer units. In some embodiments, an LbL film may include multiple copies of a particular individual single unit (e.g., a of a particular bilayer, trilayer, tetralayer, etc. unit). In some embodiments, an LbL film may include a plurality of different individual units (e.g., a plurality of distinct bilayer, trilayer, and/or tetralayer units). For example, in some embodiments, multilayer units included in an LbL film for use in accordance with the present invention may differ from one another in number of layers, materials included in layers (e.g., polymers, additives, etc.), thickness of layers, modification of materials within layers, etc. In some embodiments, an LbL film utilized in accordance with the present invention is a composite that includes a plurality of bilayer units, a plurality of tetralayer units, or any combination thereof. In some particular embodiments, an LbL film is a composite that includes multiple copies of a particular bilayer unit and multiple copies of a particular tetralayer unit.

In some embodiments, LbL films utilized in accordance with the present invention include a number of multilayer units, which is about or has a lower limit of 2, 3, 4, 5, 6, 7, 8, 9, 10, 15, 20, 25 30, 35, 40, 45, 50, 55, 60, 65, 70, 75, 80, 85, 90, 95, 100, 150, 200, 300, 400 or even 500.

LbL films may have various thickness depending on methods of fabricating and applications. In some embodiments, an LbL film has an average thickness in a range of about 1 nanometer and about 100 microns. In some embodiments, an LbL film lm has an average thickness in a range of about 1 micron and about 50 microns. In some embodiments, an LbL film has an average thickness in a range of about 2 microns and about 5 microns. In some embodiments, the average thickness of an LbL film is or more than about 1 nanometer, about 100 nanometers, about 500 nanometers, about 1 micron, about 2 microns, about 3 microns, about 4 microns, about 5 microns, about 10 microns, about 20 microns, about 50 microns, about 100 microns. In some embodiments, an LbL film has an average thickness in a range of any two values above.

In some embodiments, layers of LbL films can contain or consist of a silica material such as silicate. To give an example, Laponite® silicate clay (Lap) can be used in a multilayer film as demonstrated in Examples below.

Individual layers of LbL films can contain, be comprised of, or consist of one or more polymeric materials. In some embodiments, a polymer is degradable or non-degradable. In some embodiments, a polymer is natural or synthetic. In some embodiments, a polymer is a polyelectrolyte. In some embodiments, a polymer is a polypeptide and/or a nucleic acid. For example, a nucleic acid agent for delivery in accordance with various embodiments can serve as a layer in LbL films.

LbL films can be decomposable. In many embodiments, LbL film layers are comprised of or consisted of one or more degradable materials, such as degradable polymers and/or polyelectrolytes. In some embodiments, decomposition of LbL films is characterized by substantially sequential degradation of at least a portion of each layer that makes up an LbL film. Degradation may, for example, be at least partially hydrolytic, at least partially enzymatic, at least partially thermal, and/or at least partially photolytic. In some embodiments, materials included in degradable LbL films, and also their breakdown products, may be biocompatible, so that LbL films including them are amenable to use in vivo.

Degradable materials (e.g. degradable polymers and/or polyelectrolytes) useful in LbL films disclosed herein, include but are not limited to materials that are hydrolytically, enzymatically, thermally, and/or photolytically degradable, as well as materials that are or become degradable through application of pressure waves (e.g., ultrasonic waves).

Hydrolytically degradable polymers known in the art include for example, certain polyesters, polyanhydrides, polyorthoesters, polyphosphazenes, and polyphosphoesters. Biodegradable polymers known in the art, include, for example, certain polyhydroxyacids, polypropylfumerates, polycaprolactones, polyamides, poly(amino acids), polyacetals, polyethers, biodegradable polycyanoacrylates, biodegradable polyurethanes and polysaccharides. For example, specific biodegradable polymers that may be used include but are not limited to polylysine, poly(lactic acid) (“PLA”), poly(glycolic acid) (“PGA”), poly(caprolactone) (“PCL”), poly(lactide-co-glycolide) (“PLG”), poly(lactide-co-caprolactone) (“PLC”), and poly(glycolide-co-caprolactone) (“PGC”). Those skilled in the art will recognize that this is an exemplary, not comprehensive, list of biodegradable polymers. Of course, co-polymers, mixtures, and adducts of these polymers may also be employed.

Anionic polyelectrolytes may be degradable polymers with anionic groups distributed along the polymer backbone. Anionic groups, which may include carboxylate, sulfonate, sulphate, phosphate, nitrate, or other negatively charged or ionizable groupings, may be disposed upon groups pendant from the backbone or may be incorporated in the backbone itself. Cationic polyelectrolytes may be degradable polymers with cationic groups distributed along the polymer backbone. Cationic groups, which may include protonated amine, quaternary ammonium or phosphonium-derived functions or other positively charged or ionizable groups, may be disposed in side groups pendant from the backbone, may be attached to the backbone directly, or can be incorporated in the backbone itself. In some embodiments, Poly2, with an aliphatic backbone and a known degradation profile, as the cationic species in the PEM film.

For example, a range of hydrolytically degradable amine-containing polyesters bearing cationic side chains have been developed. Examples of these polyesters include poly(L-lactide-co-L-lysine), poly(serine ester), poly(4-hydroxy-L-proline ester), and poly[α-(4-aminobutyl)-L-glycolic acid].

In addition, poly(β-amino ester)s, prepared from the conjugate addition of primary or secondary amines to diacrylates, are suitable for use. Typically, poly(β-amino ester)s have one or more tertiary amines in the backbone of the polymer, preferably one or two per repeating backbone unit. Alternatively, a co-polymer may be used in which one of the components is a poly(β-amino ester). Poly(β-amino ester)s are described in U.S. Pat. Nos. 6,998,115 and 7,427,394, entitled “Biodegradable poly(β-amino esters) and uses thereof” and Lynn et al., J. Am. Chem. Soc. 122:10761-10768, 2000, the entire contents of both of which are incorporated herein by reference.

In some embodiments, a polymer utilized in the production of LbL film(s) can have a formula below:

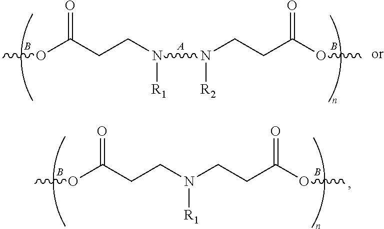

where A and B are linkers which may be any substituted or unsubstituted, branched or unbranched chain of carbon atoms or heteroatoms. The molecular weights of the polymers may range from 1000 g/mol to 20,000 g/mol, preferably from 5000 g/mol to 15,000 g/mol. In certain embodiments, B is an alkyl chain of one to twelve carbons atoms. In other embodiments, B is a heteroaliphatic chain containing a total of one to twelve carbon atoms and heteroatoms. The groups R₁ and R₂ may be any of a wide variety of substituents. In certain embodiments, R₁ and R₂ may contain primary amines, secondary amines, tertiary amines, hydroxyl groups, and alkoxy groups. In certain embodiments, the polymers are amine-terminated; and in other embodiments, the polymers are acrylated terminated. In some embodiments, the groups R₁ and/or R₂ form cyclic structures with the linker A.

Exemplary poly(β-amino esters) include

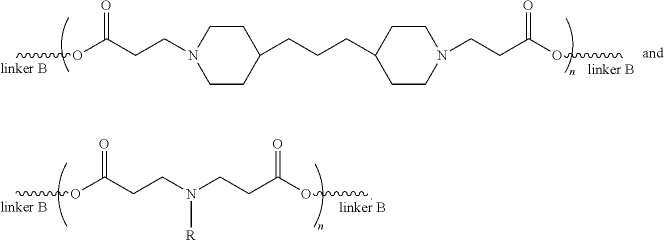

Exemplary R groups include hydrogen, branched and unbranched alkyl, branched and unbranched alkenyl, branched and unbranched alkynyl, aryl, halogen, hydroxyl, alkoxy, carbamoyl, carboxyl ester, carbonyldioxyl, amide, thiohydroxyl, alkylthioether, amino, alkylamino, dialkylamino, trialkylamino, cyano, ureido, a substituted alkanoyl group, cyclic, cyclic aromatic, heterocyclic, and aromatic heterocyclic groups, each of which may be substituted with at least one substituent selected from the group consisting of branched and unbranched alkyl, branched and unbranched alkenyl, branched and unbranched alkynyl, amino, alkylamino, dialkylamino, trialkylamino, aryl, ureido, heterocyclic, aromatic heterocyclic, cyclic, aromatic cyclic, halogen, hydroxyl, alkoxy, cyano, amide, carbamoyl, carboxylic acid, ester, carbonyl, carbonyldioxyl, alkylthioether, and thiol groups.

Exemplary linker groups B includes carbon chains of 1 to 30 carbon atoms, heteroatom-containing carbon chains of 1 to 30 atoms, and carbon chains and heteroatom-containing carbon chains with at least one substituent selected from the group consisting of branched and unbranched alkyl, branched and unbranched alkenyl, branched and unbranched alkynyl, amino, alkylamino, dialkylamino, trialkylamino, aryl, ureido, heterocyclic, aromatic heterocyclic, cyclic, aromatic cyclic, halogen, hydroxyl, alkoxy, cyano, amide, carbamoyl, carboxylic acid, ester, carbonyl, carbonyldioxyl, alkylthioether, and thiol groups. The polymer may include, for example, between 5 and 10,000 repeat units.

In some embodiments, a poly(β-amino ester)s are selected from the group consisting of

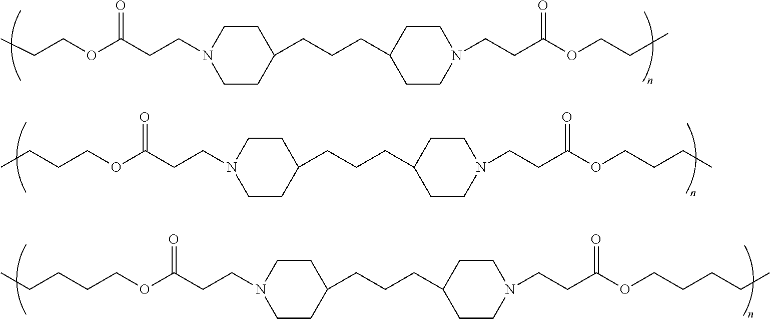

derivatives thereof, and combinations thereof.

Alternatively or additionally, zwitterionic polyelectrolytes may be used. Such polyelectrolytes may have both anionic and cationic groups incorporated into the backbone or covalently attached to the backbone as part of a pendant group. Such polymers may be neutrally charged at one pH, positively charged at another pH, and negatively charged at a third pH. For example, an LbL film may be constructed using dip coating in solutions of a first pH at which one layer is anionic and a second layer is cationic. If such an LbL film is put into a solution having a second different pH, then the first layer may be rendered cationic while the second layer is rendered anionic, thereby changing the charges on those layers.

The composition of degradable polyelectrolyte layers can be fine-tuned to adjust the degradation rate of each layer within the film, which is believe to impact the release rate of drugs. For example, the degradation rate of hydrolytically degradable polyelectrolyte layers can be decreased by associating hydrophobic polymers such as hydrocarbons and lipids with one or more of the layers. Alternatively, polyelectrolyte layers may be rendered more hydrophilic to increase their hydrolytic degradation rate. In certain embodiments, the degradation rate of a given layer can be adjusted by including a mixture of polyelectrolytes that degrade at different rates or under different conditions.

In some embodiments, polyanionic and/or polycationic layers may include a non-degradable and/or slowly hydrolytically degradable polyelectrolytes. Any non-degradable polyelectrolyte can be used. Exemplary non-degradable polyelectrolytes that could be used in thin films include poly(styrene sulfonate) (“SPS”), poly(acrylic acid) (“PAA”), linear poly(ethylene imine) (“LPEI”), poly(diallyldimethyl ammonium chloride) (“PDAC”), and poly(allylamine hydrochloride) (“PAH”).

In some embodiments, the present invention utilizes polymers that are found in nature and/or represent structural variations or modifications of such polymers that are found in nature. In some embodiments, polymers are charged polysaccharides such as, for example sodium alginate, chitosan, agar, agarose, and carragenaan. In some embodiments, polysaccharides include glycosaminoglycans such as heparin, chondroitin, dermatan, hyaluronic acid, etc. Those of ordinary skill in the art will appreciate that terminology used to refer to particular glycosaminoglycans sometimes also is used to refer to a sulfate form of the glycosaminoglycan, e.g., heparin sulfate, chondroitin sulfate, etc. It is intended that such sulfate forms are included among a list of exemplary polymers used in accordance with the present invention.

In some embodiments, an LbL film comprises at least one layer that degrades and at least one layer that delaminates. In some embodiments, a layer that degrades in adjacent a layer that delaminates. In some embodiments, an LbL film comprises at least one polycationic layer that degrades and at least one polyanionic layer that delaminates sequentially; in some embodiments, an LbL film comprises at least one polyanionic layer that degrades and at least one polycationic layer that delaminates.

In some embodiments, one or more agents is incorporated into one or more layers of an LbL film. In some embodiments, layer materials and their degradation and/or delamination characteristics are selected to achieve a desired release profile for one or more agents incorporated within the film. In some embodiments, agents are gradually, or otherwise controllably, released from an LbL film. In some embodiments, agents are or comprise, but are not limited to, for example, therapeutic agents, cytotoxic agents, diagnostic agents, prophylactic agents, nutraceutical agents, nucleic acids, proteins, peptides, lipids, carbohydrates, hormones, metals, radioactive elements and compounds, drugs, vaccines, immunological agents, and/or combinations thereof. In some embodiments, multiple agents are associated with a thin film by a conjugate. In some embodiments, agents are in a conjugate associated with a thin film while other agents are also incorporated into the thin film. In some embodiments, a same agent in a conjugate associated with a thin film is also incorporated as an agent into the thin film.

In accordance with the present invention, LbL films may be exposed to a liquid medium (e.g., intracellular fluid, interstitial fluid, blood, intravitreal fluid, intraocular fluid, gastric fluids, etc.). In some embodiments, layers of LbL films degrade and/or delaminate in such a liquid medium. In some embodiments, such degradation and/or delamination achieves delivery of one or more agents, for example according to a predetermined release profile.

In some embodiments, assembly of an LbL film may involve a series of dip coating steps in which a substrate is dipped in alternating solutions. In some embodiments, LbL assembly of a film may involve mixing, washing or incubation steps to facilitate interactions of layers, in particular, for non-electrostatic interactions. Additionally or alternatively, it will be appreciated that an LbL film may also be achieved by spray coating, dip coating, brush coating, roll coating, spin casting, or combinations of any of these techniques. In some embodiments, spray coating is performed under vacuum. In some embodiments, spray coating is performed under vacuum of about 10 pounds per square inch, 20 pounds per square inch, 50 pounds per square inch, 100 pounds per square inch, 200 pounds per square inch or 500 pounds per square inch. In some embodiments, spray coating is performed under vacuum in a range of any two values above.

In light of this provided demonstration that effective delivery of small molecules can be achieved using LbL films, those of ordinary skill in the art will appreciate that various embodiments and variations of the exemplified compositions can now be prepared that will similarly achieve effective small molecule delivery. Certain characteristics of compositions described herein may be modulated to achieve desired functionalities for different applications.

In some embodiments, loading capacity may be modulated, for example, by changing the number of multilayer units that make up the film, the type of degradable polymers used, the type of polyelectrolytes used, and/or concentrations of solutions of agents used during construction of LbL films.

Additionally or alternatively, other conditions for example prior to or during deposition can be adjusted as those of ordinary skills in the art would appreciate and understand. In some embodiments, suitable pH values can include 2, 3, 4, 5, 6, 7, 8, 9, 10. In some embodiments, a suitable salt concentration is less than 5 mol/liter, 1 mol/liter, 0.5 mol/liter, 0.1 mol/liter, and 0.01 mol/liter. In some embodiments, suitable buffers include sodium acetate, Tris HCl, HEPES, Glycine, sodium phosphate or combination thereof.

Similarly, in some embodiments, release kinetics (both rate of release and release timescale of an agent) may be modulated by changing any or a combination of aforementioned factors.

### Growth Factor

In some embodiments, a composite device releases growth factor over time. In some embodiments, a porous polymer membrane is associated with growth factor. In some embodiments, an LbL film is associated with growth factor. In some embodiments, a porous polymer membrane is associated with multiple growth factors. In some embodiments, an LbL film is associated with multiple growth factors. In some embodiments, composite devices degrade, decompose, and/or delaminate releasing growth factor(s) at a defect site. In some embodiments, multiple growth factors are released in a cascade of growth factor mimicking a natural bone repair and regeneration process.

In some embodiments, BMP, PDGF, VEGF, and/or PIGF can be impregnated within a porous polymer membrane and/or a degradable LbL film and release into a defect site.

In the context of bone tissue engineering, bone morphogenetic protein (BMP) and platelet derived growth factor (PDGF) are two of the most prominent growth factors introduced to the clinic in recent years for the treatment of defects in bone presenting as orthopedic and oral and maxillofacial problems. A biological interface on the membrane surface is created by applying a multilayer thin film to the membrane composed of osteoinductive BMP (e.g. BMP-2) and angiogenic/mitogenic PDGF (e.g. PDGF-BB) factors. In some embodiments, PDGF and BMP mimic a wound healing cascade. In some embodiments, top layers of PDGF mimics neovascularization. In some embodiments, bottom layers of BMP mimics bone growth. In some embodiments, hydrolytically degradable poly(β-amino ester), Poly2, and poly(acrylic acid) (PAA), caused PDGF-BB to quickly release, followed by a more sustained release of BMP-2.

In some embodiments, growth factor known in the art include, for example, adrenomedullin, angiopoietin, autocrine motility factor, basophils, brain-derived neurotrophic factor, bone morphogenetic protein, colony-stimulating factors, connective tissue growth factor, endothelial cells, epidermal growth factor, erythropoietin, fibroblast growth factor, fibroblasts, glial cell line-derived neurotrophic factor, granulocyte colony stimulating factor, granulocyte macrophage colony stimulating factor, growth differentiation factor-9, hepatocyte growth factor, hepatoma-derived growth factor, insulin-like growth factor, interleukins, keratinocyte growth factor, keratinocytes, lymphocytes, macrophages, mast cells, myostatin, nerve growth factor, neurotrophins, platelet-derived growth factor, placenta growth factor, osteoblasts, platelets, proinflammatory, stromal cells, T-lymphocytes, thrombopoietin, transforming growth factor alpha, transforming growth factor beta, tumor necrosis factor-alpha, vascular endothelial growth factor and combinations thereof.

Some embodiments of the present invention can be particularly useful for healing bone and/or tissue defects. Exemplary agents useful as growth factor for defect repair and/or healing can include, but are not limited to, growth factors for defect treatment modalities now known in the art or later-developed; exemplary factors, agents or modalities including natural or synthetic growth factors, cytokines, or modulators thereof to promote bone and/or tissue defect healing. Suitable examples may include, but not limited to 1) topical or dressing and related therapies and debriding agents (such as, for example, Santyl® collagenase) and Iodosorb® (cadexomer iodine); 2) antimicrobial agents, including systemic or topical creams or gels, including, for example, silver-containing agents such as SAGs (silver antimicrobial gels), (CollaGUARD™, Innocoll, Inc) (purified type-I collagen protein based dressing), CollaGUARD Ag (a collagen-based bioactive dressing impregnated with silver for infected wounds or wounds at risk of infection), DermaSIL™ (a collagen-synthetic foam composite dressing for deep and heavily exuding wounds); 3) cell therapy or bioengineered skin, skin substitutes, and skin equivalents, including, for example, Dermograft (3-dimensional matrix cultivation of human fibroblasts that secrete cytokines and growth factors), Apligraf® (human keratinocytes and fibroblasts), Graftskin® (bilayer of epidermal cells and fibroblasts that is histologically similar to normal skin and produces growth factors similar to those produced by normal skin), TransCyte (a Human Fibroblast Derived Temporary Skin Substitute) and Oasis® (an active biomaterial that comprises both growth factors and extracellular matrix components such as collagen, proteoglycans, and glycosaminoglycans); 4) cytokines, growth factors or hormones (both natural and synthetic) introduced to the wound to promote wound healing, including, for example, NGF, NT3, BDGF, integrins, plasmin, semaphoring, blood-derived growth factor, keratinocyte growth factor, tissue growth factor, TGF-alpha, TGF-beta, PDGF (one or more of the three subtypes may be used: AA, AB, and B), PDGF-BB, TGF-beta 3, factors that modulate the relative levels of TGFβ3, TGFβ1, and TGFβ2 (e.g., Mannose-6-phosphate), sex steroids, including for example, estrogen, estradiol, or an oestrogen receptor agonist selected from the group consisting of ethinyloestradiol, dienoestrol, mestranol, oestradiol, oestriol, a conjugated oestrogen, piperazine oestrone sulphate, stilboestrol, fosfesterol tetrasodium, polyestradiol phosphate, tibolone, a phytoestrogen, 17-beta-estradiol; thymic hormones such as Thymosin-beta-4, EGF, HB-EGF, fibroblast growth factors (e.g., FGF1, FGF2, FGF7), keratinocyte growth factor, TNF, interleukins family of inflammatory response modulators such as, for example, IL-10, IL-1, IL-2, IL-6, IL-8, and IL-10 and modulators thereof; INFs (INF-alpha, -beta, and -delta); stimulators of activin or inhibin, and inhibitors of interferon gamma prostaglandin E2 (PGE2) and of mediators of the adenosine 3′,5′-cyclic monophosphate (cAMP) pathway; adenosine A1 agonist, adenosine A2 agonist or 5) other agents useful for wound healing, including, for example, both natural or synthetic homologues, agonist and antagonist of VEGF, VEGFA, IGF; IGF-1, proinflammatory cytokines, GM-CSF, and leptins and 6) IGF-1 and KGF cDNA, autologous platelet gel, hypochlorous acid (Sterilox® lipoic acid, nitric oxide synthase3, matrix metalloproteinase 9 (MMP-9), CCT-ETA, alphavbeta6 integrin, growth factor-primed fibroblasts and Decorin, silver containing wound dressings, Xenaderm™, papain wound debriding agents, lactoferrin, substance P, collagen, and silver-ORC, placental alkaline phosphatase or placental growth factor, modulators of hedgehog signaling, modulators of cholesterol synthesis pathway, and APC (Activated Protein C), keratinocyte growth factor, TNF, Thromboxane A2, NGF, BMP bone morphogenetic protein, CTGF (connective tissue growth factor), wound healing chemokines, decorin, modulators of lactate induced neovascularization, cod liver oil, placental alkaline phosphatase or placental growth factor, and thymosin beta 4. In certain embodiments, one, two three, four, five or six agents useful for wound healing may be used in combination. More details can be found in U.S. Pat. No. 8,247,384, the contents of which are incorporated herein by reference.

It is to be understood that agents useful for growth factor for healing (including for example, growth factors and cytokines) above encompass all naturally occurring polymorphs (for example, polymorphs of the growth factors or cytokines). Also, functional fragments, chimeric proteins comprising one of said agents useful for wound healing or a functional fragment thereof, homologues obtained by analogous substitution of one or more amino acids of the wound healing agent, and species homologues are encompassed. It is contemplated that one or more agents useful for wound healing may be a product of recombinant DNA technology, and one or more agents useful for wound healing may be a product of transgenic technology. For example, platelet derived growth factor may be provided in the form of a recombinant PDGF or a gene therapy vector comprising a coding sequence for PDGF.

### Agent for Delivery

In some embodiments, one or more agents is incorporated into one or more layers of a porous polymer membrane and/or an LbL film. In some embodiments, a porous polymer membrane comprises an agent for delivery. In some embodiments, an LbL includes an agent for delivery. In some embodiments, layer materials and their degradation, decomposition, and/or delamination characteristics are selected to achieve a desired release profile for one or more agents incorporated within a porous polymer membrane and/or LbL film. In some embodiments, agents are gradually, or otherwise controllably, released.

In some embodiments, agents are or comprise, but are not limited to, for example, small molecules. In some embodiments, agents are or comprise, but are not limited to, for example, therapeutic agents, cytotoxic agents, diagnostic agents, prophylactic agents, nutraceutical agents, nucleic acids, proteins, peptides, lipids, carbohydrates, hormones, metals, radioactive elements and compounds, drugs, vaccines, immunological agents, and/or combinations thereof. Exemplary agents include, but are not limited to, for example, therapeutic agents, cytotoxic agents, diagnostic agents (e.g. contrast agents; radionuclides; and fluorescent, luminescent, and magnetic moieties), prophylactic agents (e.g. vaccines), nutraceutical agents (e.g. vitamins, minerals, etc.), nucleic acids (e.g., siRNA, RNAi, and microRNA agents), proteins (e.g. antibodies), peptides, lipids, carbohydrates, hormones, metals, radioactive elements and compounds, drugs, vaccines, immunological agents, etc., and/or combinations thereof may be associated with a porous polymer membrane and/or LbL film as disclosed herein. In some embodiments, an agent for delivery is a small molecule.

In some embodiments, a therapeutic agent is or comprises an anti-viral agent, anesthetic, anticoagulant, anti-cancer agent, inhibitor of an enzyme, steroidal agent, anti-inflammatory agent, anti-neoplastic agent, antigen, vaccine, antibody, decongestant, antihypertensive, sedative, birth control agent, progestational agent, anti-cholinergic, analgesic, anti-depressant, anti-psychotic, β-adrenergic blocking agent, diuretic, cardiovascular active agent, vasoactive agent, anti-glaucoma agent, neuroprotectant, angiogenesis inhibitor, antibiotics, NSAIDs, glaucoma medications, angiogenesis inhibitors, and/or neuroprotective agents, etc.

In some embodiments, a small molecule has a low molecular weight. In some embodiments, a low molecular weight being below about 100 Da, 200 Da, 300 Da, 400 Da, 0.5 kDa, 1 kDa, 1.5 kDa, 2 kDa, 3 kDa, 4 kDa, 5 kDa, 6 kDa, 7 kDa, 8 kDa, 9 kDa, or 10 kDa.

In some embodiments, a small molecule has pharmaceutical activity. In some embodiments, a small molecule is a clinically-used drug. In some embodiments, a small molecule is or comprises an antibiotic, anti-viral agent, anesthetic, anticoagulant, anti-cancer agent, inhibitor of an enzyme, steroidal agent, anti-inflammatory agent, anti-neoplastic agent, antigen, vaccine, antibody, decongestant, antihypertensive, sedative, birth control agent, progestational agent, anti-cholinergic, analgesic, anti-depressant, anti-psychotic, β-adrenergic blocking agent, diuretic, cardiovascular active agent, vasoactive agent, anti-glaucoma agent, neuroprotectant, angiogenesis inhibitor, etc.

In some embodiments, a small molecule may be an antibiotic. A non-exclusive list of antibiotics may include, but is not limited to, β-lactam antibiotics, macrolides, monobactams, rifamycins, tetracyclines, chloramphenicol, clindamycin, lincomycin, fusidic acid, novobiocin, fosfomycin, fusidate sodium, capreomycin, colistimethate, gramicidin, minocycline, doxycycline, bacitracin, erythromycin, nalidixic acid, vancomycin, gentamicin, and trimethoprim. For example, β-lactam antibiotics can be ampicillin, aziocillin, aztreonam, carbenicillin, cefoperazone, ceftriaxone, cephaloridine, cephalothin, cloxacillin, moxalactam, penicillin G, piperacillin, ticarcillin and any combination thereof.

In some embodiments, a small molecule may be or comprise an anti-inflammatory agent. A non-exclusive list of anti-inflammatories may include, but is not limited to, corticosteroids (e.g., glucocorticoids), cycloplegics, non-steroidal anti-inflammatory drugs (NSAIDs), immune selective anti-inflammatory derivatives (ImSAIDs), and any combination thereof. Exemplary NSAIDs include, but not limited to, celecoxib (Celebrex®); rofecoxib (Vioxx®), etoricoxib (Arcoxia®), meloxicam (Mobic®), valdecoxib, diclofenac (Voltaren®, Cataflam®), etodolac (Lodine®), sulindac (Clinori®), aspirin, alclofenac, fenclofenac, diflunisal (Dolobid®), benorylate, fosfosal, salicylic acid including acetylsalicylic acid, sodium acetylsalicylic acid, calcium acetylsalicylic acid, and sodium salicylate; ibuprofen (Motrin), ketoprofen, carprofen, fenbufen, flurbiprofen, oxaprozin, suprofen, triaprofenic acid, fenoprofen, indoprofen, piroprofen, flufenamic, mefenamic, meclofenamic, niflumic, salsalate, rolmerin, fentiazac, tilomisole, oxyphenbutazone, phenylbutazone, apazone, feprazone, sudoxicam, isoxicam, tenoxicam, piroxicam (Feldene®), indomethacin (Indocin®), nabumetone (Relafen®), naproxen (Naprosyn®), tolmetin, lumiracoxib, parecoxib, licofelone (ML3000), including pharmaceutically acceptable salts, isomers, enantiomers, derivatives, prodrugs, crystal polymorphs, amorphous modifications, co-crystals and combinations thereof.

Those skilled in the art will recognize that this is an exemplary, not comprehensive, list of small molecules that can be released using compositions and methods in accordance with the present disclosure. In addition to a therapeutic agent or alternatively, various other agents may be associated with a coated substrate in accordance with the present disclosure.

### EXEMPLIFICATION

### Example 1

### Materials and Methods

Alendronate sodium trihydrate (Alfa-Aesar), PLGA (50:50) (MW˜38,000-54,000), PAA (Mv˜450,000) (Sigma) and PDGF-BB (Osteohealth) were purchased. Poly2 (Mn˜12,000) was synthesized using a previously reported method in Lynn D M, Langer R. “Degradable poly(beta-amino esters): Synthesis, characterization, and self-assembly with plasmid DNA” 122 Journal of the American Chemical Society 44, 10761-10768 (2000), which is incorporated by reference in its entirety herein. BMP-2 (Pfizer) was obtained through a materials transfer agreement.

### PLGA Membrane Formation and Alendronate Conjugation.

The PLGA membrane was prepared using the diffusion induced phase separation process. A homogenous 20 wt % solution of PLGA in dimethylformamide (DMF) was prepared at room temperature and degassed. Using a doctor blade knife, the polymer solution was cast on a glass plate and immersed in deionized water at room temperature. The resulting membrane was rinsed continuously with deionized (DI) water for 2 hours, immersed in DI water for an additional 48 hours and dried at ambient conditions. A micrometer was used to determine the composite membrane thickness by measuring at least 10 different locations including the center. Alendronate was conjugated to PLGA using a modified version of a previously reported procedure reported in Wang D, Miller S, Sima M, Kopeckova P, Kopecek J., “Synthesis and evaluation of water-soluble polymeric bone-targeted drug delivery systems,” 14 Bioconjugate Chemistry 5, 853-859 (2003), which is incorporated by reference in its entirety herein.

As a representative synthetic procedure, 1 g of PLGA was dissolved in 15 mL of dichloromethane, and added to 15 milligrams of p-nitrophenyl chloroformate and 10 microliters of pyridine to activate the terminal hydroxyl group of the polymer, corresponding to about 10 wt % functionalization. The reaction was carried out for 4 hours in an ice bath under inert atmosphere. The resulting solution was further diluted by the addition of 10 mL of dichloromethane, and subsequently extracted with 0.1% HCl and brine. After separation, the organic phase was dried over magnesium sulfate, filtered and evaporated to yield activated PLGA polymer. Activated PLGA was dissolved in 5 milliliters of DMF, and treated with 10 mg of alendronate and 5 microliters of triethylamine (mixed prior to addition) for 24 hours at room temperature under inert atmosphere. After 24 hours, the reaction mixture was precipitated in cold ether, washed with water, filtered and vacuum dried. Additional dialysis was carried out for 48 hours to remove free alendronate through 6K MWCO membrane. 31P-NMR was carried out on the dialyzed product to confirm conjugation.

### PEM Deposition, Characterization and Release.

Multilayer films were deposited using the LbL method. Sodium acetate buffer (0.1 M, pH 4.0) was used for preparing polyelectrolyte solutions. Polyelectrolyte solutions were prepared at 1 mg/ml (PAA, Poly2). Concentrations of PDGF-BB and BMP-2 dipping solutions were adjusted to control the total loading in the PEMs. PLGA membranes 2 cm×4 cm were sterilized using plasma treatment with air for 5 seconds using a Harrick PDC-32G plasma cleaner (Harrick Plasma) on high RF power and immediately immersed in Poly2 solution. Layers were deposited using a Carl Zeiss HMS-DS50 slide stainer. The substrate was immersed alternatively in Poly2 (5 min), PAA (5 min), either BMP-2 or PDGF-BB (5 min) and PAA (5 min). There were 3 wash steps of 10 s, 20 s and 30 s in deionized water between each polyelectrolyte solution. The cycle was iterated 40 times with each growth factor. An 8 mm hollow punch (Mayhew Pro) was used to produce circular test samples from the rectangular membrane. Films were characterized using a JEOL 6700 Field Emission Scanning Electron Microscope. For in vitro release experiments, coated membranes were incubated in 1 ml cell culture media (α-MEM supplemented with 20% FBS, 1% penicillin-streptomycin solution) at 37 degrees Celsius. The release medium was changed at pre-determined time points and assayed for BMP-2 and PDGF-BB using ELISA (Peprotech).

### In Vivo Critical Size Defect Studies.

All animal work was performed in accordance with protocols approved by the Committee on Animal Care (IACUC) at MIT. Animals were cared for in an AAALAC certified MIT animal facility meeting federal, state, local, and NIH guidelines for animal care. Skeletally mature adult male Sprague-Dawley rats (350-400 grams; Charles River) were used in the study. Soft tissue dissection after a scalp incision was used to expose the calvarium. The periosteium was scraped off to expose the underlying bone. A trephine drill (Salvin Dental Specialties) was used to create a circular critical size defect (8.0 millimeters diameter) with intermittent irrigation of the site with phosphate buffer saline. The calvarium was excised and discarded while maintaining the dura and a PLGA membrane (8.0 millimeters diameter) was placed on the defect site and immobilized with sutures to the surrounding soft tissue. The wound and incision were closed and animals were provided with analgesics until recovery.

### μCT Analysis and Histology Evaluation.

Anesthetized live animals were imaged with a μCT (eXplore CT120, GE Medical Systems). Scanning protocol: Shutter speed (325 s), 2×2 binning, 70 kV, 50 mA, 220 images, 0.877o increments, gain: 100 and offset: 20. Images were reconstructed and analyzed with MicroView (GE Healthcare). For each animal at each time point, a three-dimensional computed tomographic reconstruction was created. Defect margins were established to delineate a standard region of interest (ROI) per animal. A threshold value (constant for all groups) was selected and the bone mineral density (BMD) and bone volume (BV) were measured using the Bone Analysis tool. After euthanasia at pre-determined time points, calvaria were excised and fixed in 4% paraformaldehyde (PFA) for 48 hours and transferred to a 70% ethanol solution. Calvaria were partially decalcified for about 4 hours using a rapid decalcifying formic acid/hydrochloric acid mixture (Decalcifying Solution, VWR). The defect area was cut in cross-section with a razor blade and embedded in paraffin wax. Sections (5 microns) of the cross section were stained with Masson's trichrome stain and imaged using brightfield microscopy.

### Mechanical Testing of Calvaria

Explanted calvaria were stored in PBS for immediate mechanical compression testing (Instron 5943). The thickness of the calvaria was measured using a set of calipers before and after applying a constant force of 10 N for 60 seconds. Stiffness was calculated using the formula:

$$Stiffness = \frac{Force}{Change in thickness} \qquad ( 1 )$$

The compressive failure force, perpendicular to the regenerated calvarial bone, was the maximum load achieved before compressive fracture.

### Statistical Analysis.

Prism 5 (GraphPad) was used for all analyses. Results are presented as means±SEM. Data were analyzed by ANOVA and comparisons were performed with a Tukey post hoc test (multiple groups). p<0.05 was considered significant.

### Example 2

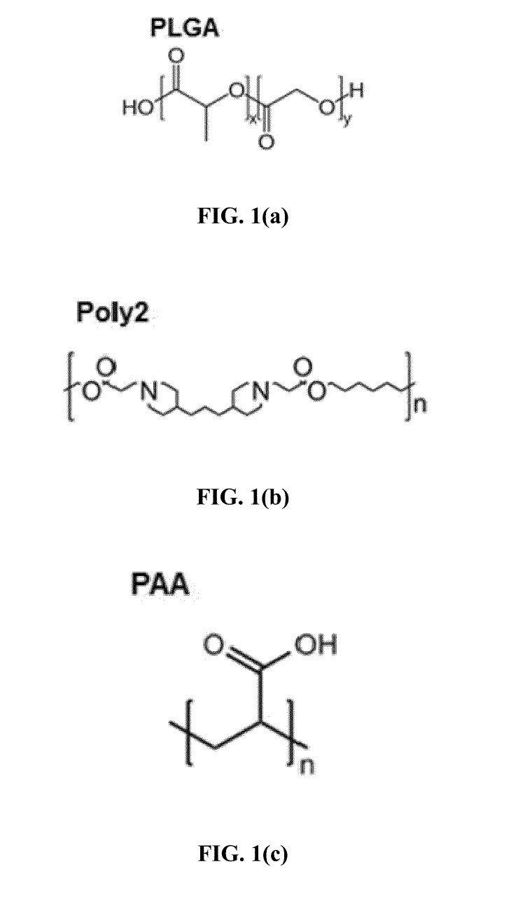

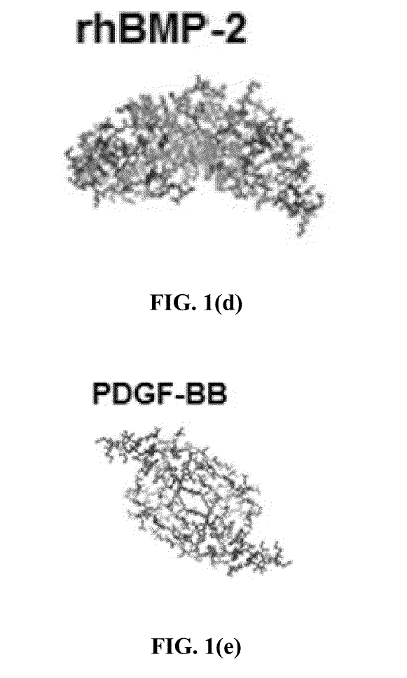

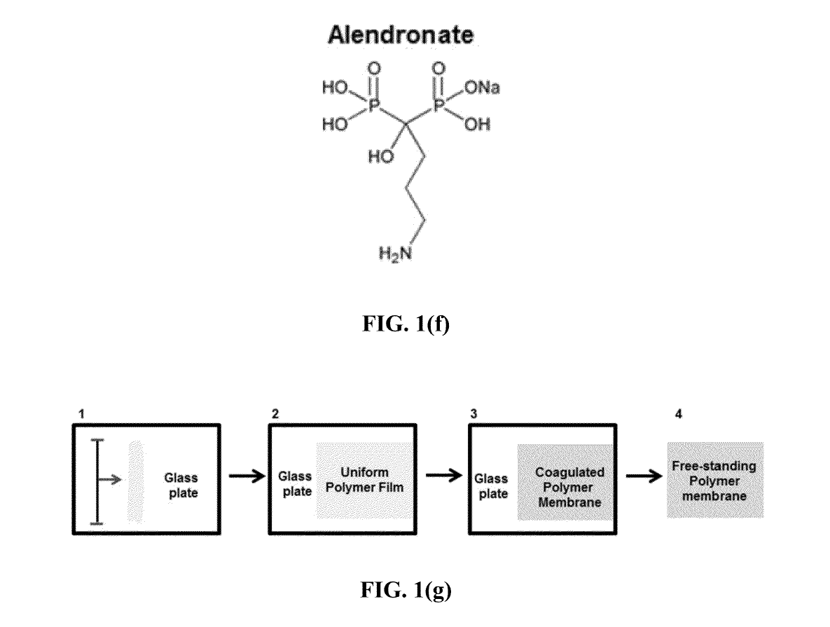

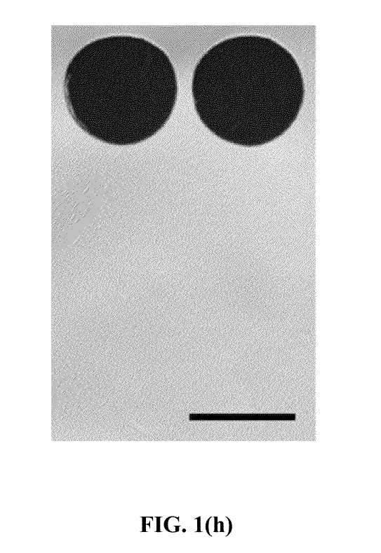

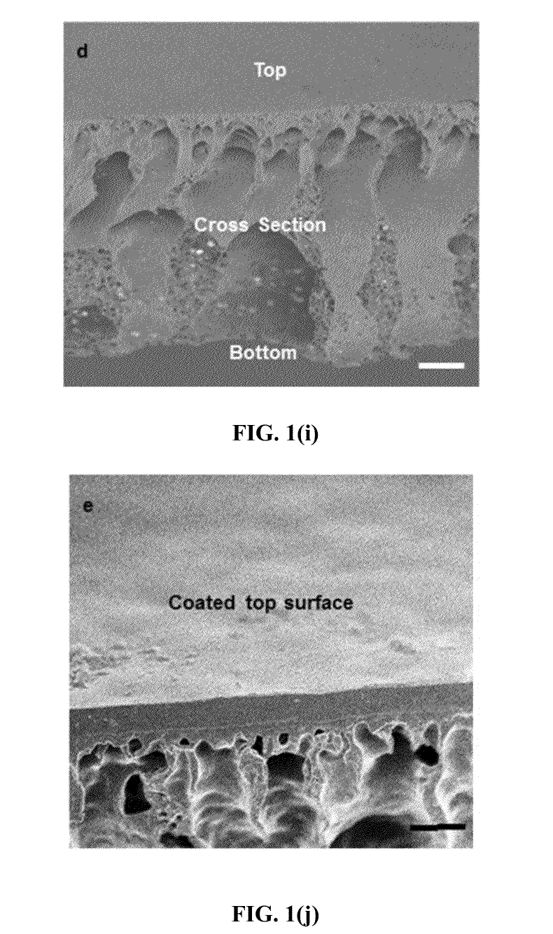

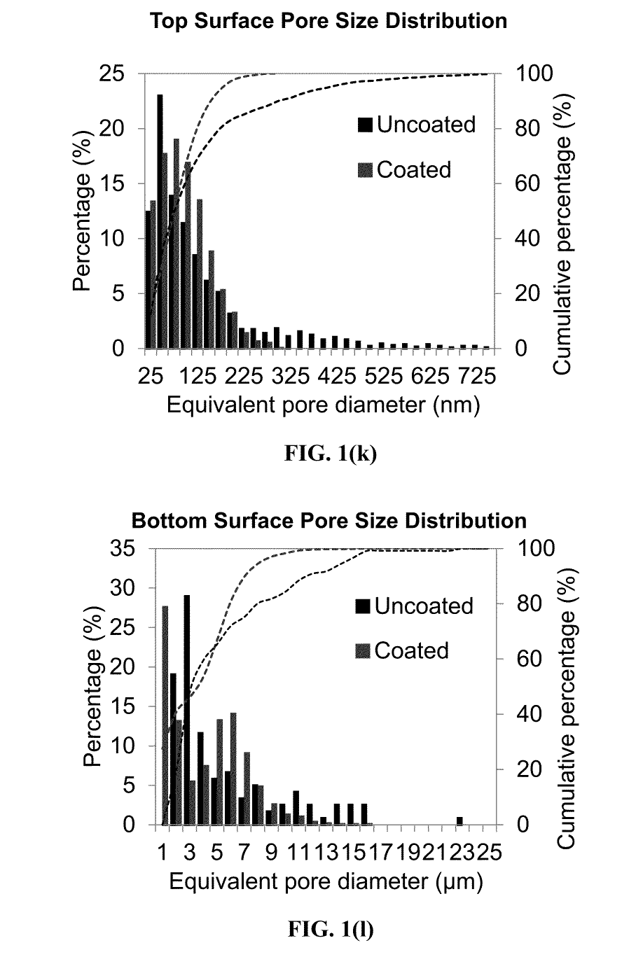

A composite device consisting of a biodegradable porous ultrathin multilayer polymer to repair a CMF defect was created consisting of a poly(lactic-co-glycolic) acid (PLGA) membrane with defined physical properties such as thickness and surface morphology. FIGS. 1(a)-(f) show the molecular structures of materials in the system, including, hydrophobic PLGA, which is used to form the membrane and Poly2, PAA, BMP-2, and PDGF-BB are part of the bioactive interface that initiates a bone wound healing cascade. A bisphosphonate molecule, alendronate, is conjugated to PLGA. This bulk polymer membrane was cut and customized to the size of the wound prior to application, allowing it to induce targeted bone repair. The polymer membrane had microstructures with interconnected pores that allowed for association and sequestration of active biologics and support for active cell proliferation for vascular and bone tissue. It was hypothesized that controlled growth factor delivery from the membrane would (i) recapitulate cellular regenerative processes and substantially enhance bone formation by inducing angiogenesis followed by osteogenesis and, (ii) promote rapid bone repair and provide a supporting structure to guide the regenerative process where needed. A biological interface was created on the membrane surface by applying a multilayer thin film to the membrane composed of osteoinductive (BMP-2) and angiogenic/mitogenic (PDGF-BB) factors. A hydrolytically degradable poly(β-amino ester), Poly2, and poly(acrylic acid) (PAA), that caused PDGF-BB to release quickly, followed by a more sustained release of BMP-2. To potentially enhance bone formation by modulating bone resorption, alendronate, a bisphosphonate that binds to the mineral phase of osseous tissue and is an inhibitor of osteoclast resorption of bone, was used. By changing the components of the system, structure-function relationships were elucidated and provided insight into the formation of bone and optimal design. It was demonstrated that this composite device allowed direct control of the bone regenerative process to rapidly induce repair in a critical size rat calvaria defect with mechanically competent bone. This materials based approach provides a new alternative to autologous bone grafting or the use of transplanted stem cells for CMF bone repair and reconstruction.

### Polymer “Skin” Construction

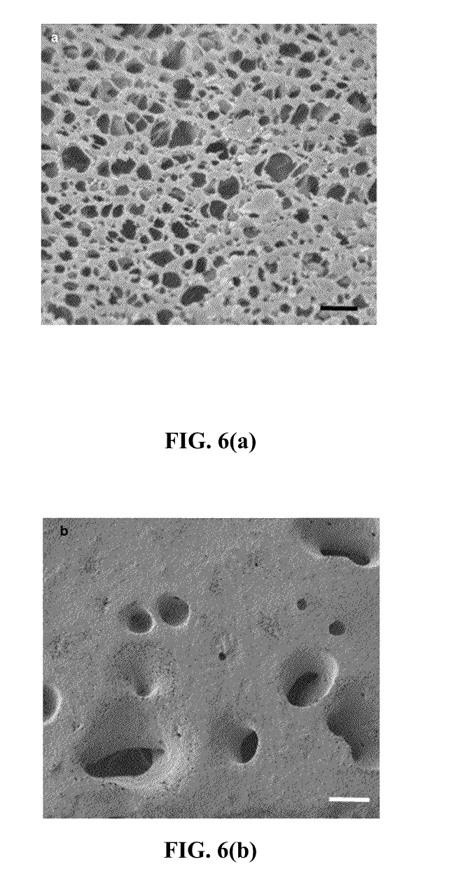

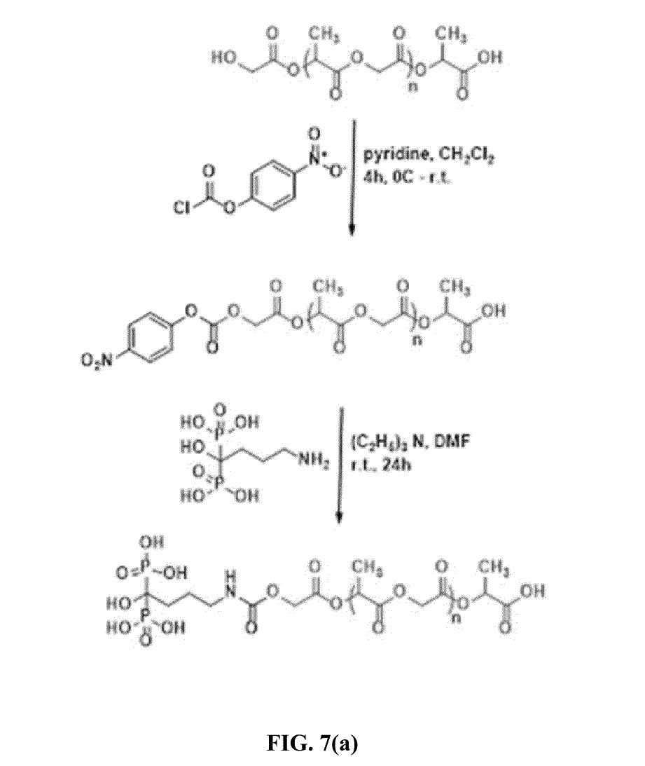

Porous PLGA scaffolds were fabricated using a solvent induced phase inversion technique to obtain a flexible polymer membrane. See for example, Graham P D, Brodbeck K J, McHugh A J, “Phase inversion dynamics of PLGA solutions related to drug delivery,” 58 J Control Release 2, 233-245 (1999); Lo H, Ponticiello M S, Leong K W, “Fabrication of controlled release biodegradable foams by phase separation,” 1 Tissue Eng 1, 15-28 (1995); and Mikos A G, Thorsen A J, Czerwonka L A, Bao Y, Langer R, Winslow D N, et al., “Preparation and Characterization of Poly(L-Lactic Acid) Foams,” 35 Polymer 5, 1068-1077 (1994), which are incorporated by reference in their entirety herein. Solvent induced phase inversion has been successfully used to develop asymmetric separation membranes for diverse applications including filtration, gas separation, biological reactors and cell-seeded scaffolds. Solvent induced phase inversion was used to create a degradable polymeric membrane with a hierarchical architecture with tunable surface chemistry to initiate vascularization, migration, differentiation and osteogenesis. An asymmetric membrane was created using diffusion induced phase separation of a ternary system of PLGA-DMF-water. FIG. 1(g) is a schematic of a phase-inversion membrane formation process. FIG. 1(g) panel (1) shows a PLGA-DMF solution poured on a glass plate. FIG. 1(g) panel (2) shows a doctor blade having been used to spread a polymer solution uniformly on the glass plate. FIG. 1(g) panel (3) shows a polymer solution immersed into a deionized water bath. FIG. 1(g) panel (4) shows a resultant film detached from a glass substrate. FIG. 1(h) shows a macroscopic image of a porous polymer membrane structure resulting in a uniform polymer support (scale bar, 8 millimeters). FIG. 1(i) shows a scanning electron micrographs demonstrating a highly ordered cross section (scale bar, 10 microns). FIG. 1(h) shows a cross section of the porous polymer membrane. FIG. 6 shows a scanning electron micrographs of a porous polyment membrane surface. FIG. 6(a) shows an SEM image of a top surface (scale bar, 1 microns). FIG. 6(b) bottom surface (scale bar, 100 microns). The SEM images confirmed pores increasing in size along a thickness of a porous polymer membrane, varying from about 2 microns on a top surface to 200 microns on a bottom surface.

The side in direct contact with the glass plate (bottom surface) during phase inversion had a broad pore size distribution, that spanned ˜1.5 μm to ˜20 μm. FIG. 1(k) is a bar graph illustrating pore size distribution for the top surface of an example membrane (the surface away from the glass plate). The top surface (away from the glass plate) had much smaller pore sizes that were less than 300 nm. The data indicate that there is a general trend of smaller pore sizes on the top surface and larger pore sizes on the bottom surface. FIG. 1(l) is a bar graph illustrating pore size distribution for the bottom surface or an example membrane (the surface in contract with the glass plate). Scanning electron micrographs (SEM) of PLGA membranes coated with growth factors revealed a conformal, single coating on the membrane and within the internal structure; typically the coating thickness was ˜0.5 μm for single growth factor and ˜1 μm for dual growth factor coatings. The thickness of the PEM coatings reduces pore size and shifts the pore size distribution, as expected. Over 95% of the nanoscale pores on the top surface, smaller than the thickness of the coating, were covered. The porosity was estimated by dividing the total area of the pores by the total area of the image. As anticipated, the porosity of the uncoated and coated bottom surface remained between 30-40%, whereas porosity of the top surface was 26% and 8% for the uncoated and coated top surface respectively—a consequence of reduced pore area due to the PEM coating.

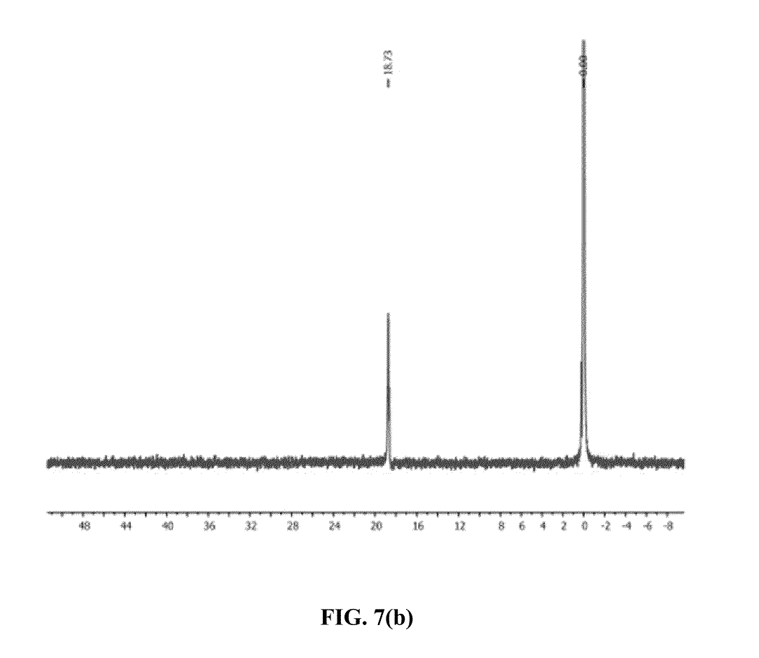

PLGA with end-groups conjugated with alendronate were also used. Alendronate has a high affinity for hydroxyapatite and is used in the clinical management of osteoporosis. Alendronate has also been combined with various nanoparticles that act as drug carriers targeting bone tissue. A PLGA terminal hydroxyl group was activated with p-nitrophenyl chloroformate to generate a highly efficient chloroformate leaving group at the end of PLGA chain, which was quantitatively substituted by the alendronate amine group. FIG. 7(a) shows conjugation of PLGA with alendronate. FIG. 7(b) shows product characterization Phosphorus-31-NMR if the conjugated PLGA-alendronate product. FIG. 7(b) graphed the P signal at 18.7 ppm relative to phosphoric acid standard corresponding to alendronate phosphonate moiety. As shown, the reaction placed negatively charged phosphonate end groups at the end of the hydrophobic PLGA backbone, at 2.07±0.33 μg (as measured by SEM) alendronate per mg of polymer, essentially generating an amphiphilic molecule. During phase inversion, PLGA precipitated during water immersion to form a membrane in aqueous solution; for end-modified PLGA, the alendronate moiety extended towards the hydrophilic environment, and made the alendronate accessible on the surface of the membrane. The alendronate groups were able to bind to hydroxyapatite, and thus inhibited bone resorption and potentially leading to rapid bone formation. Unmodified membranes and alendronate conjugated PLGA membranes are denoted as M and M_{Al} respectively.

The porous polymer membrane surface was coated using PEM, which are nanostructured films formed by an LbL technique of iterative adsorption of alternately charged materials. See for example, Decher G., “Fuzzy Nanoassemblies: Toward Layered Polymeric Multicomposites,” 277 Science, 1232 (1997); Hammond P T, “Form and function in multilayer assembly: New applications at the nanoscale,” 16 Advanced Materials 15, 1271-1293 (2004); and Boudou T, Crouzier T, Ren K, Blin G, Picart C., “Multiple functionalities of polyelectrolyte multilayer films: new biomedical applications,” 22 Adv Mater 4, 441-467 (2010). PEMs can sequester and elute multiple biologic cargos in a controlled, pre-programmed manner over several weeks; the release profiles can be easily tuned by modifying the multilayer architecture. Poly2 was used with an aliphatic backbone and a known degradation profile, as the cationic species in the PEM film. An LbL film composition consisted of Poly2, PAA, and a growth factor (PDGF or BMP-2) in a tetralayer repeat unit [Poly2/PAA/PDGF-BB/PAA] or [Poly2/PAA/BMP-2/PAA] denoted as P and B respectively. A subscript indicates the total dose of each growth factor in micrograms. BMP-2 containing layers were deposited directly on a porous polymer membrane surface. Subsequently, PDGF-BB containing layers were deposited on top of BMP-2 containing layers. FIG. 1(j) shows a PLGA membrane coated with [Poly2/PAA/rhBMP-2/PAA]₄₀+[Poly2/PAA/rhPDGF-BB/PAA]₄₀ (scale bar, 2 microns) (The subscript indicates the total dose of each growth factor in micrograms). Scanning electron micrographs (SEM) of PLGA membranes coated with growth factors revealed a conformal, single film on the membrane and the internal microstructure. Typically the film thickness was about 0.5 microns for single growth factor and about 1 micron for dual growth factor films.

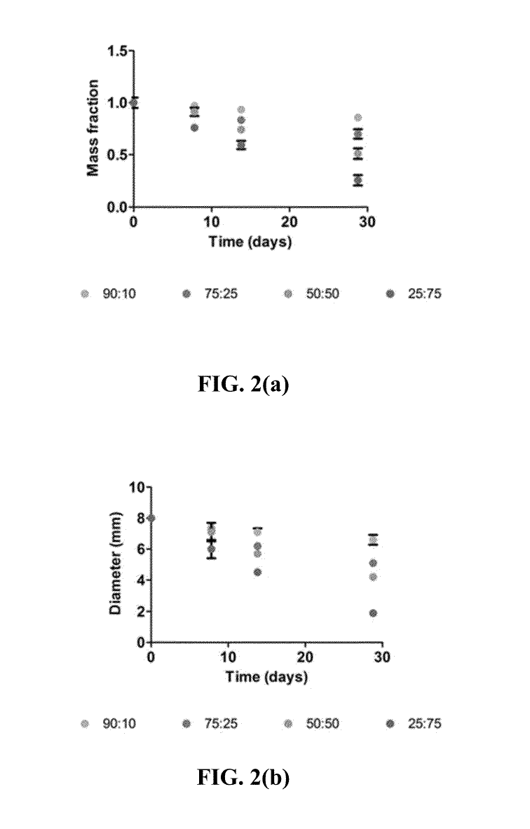

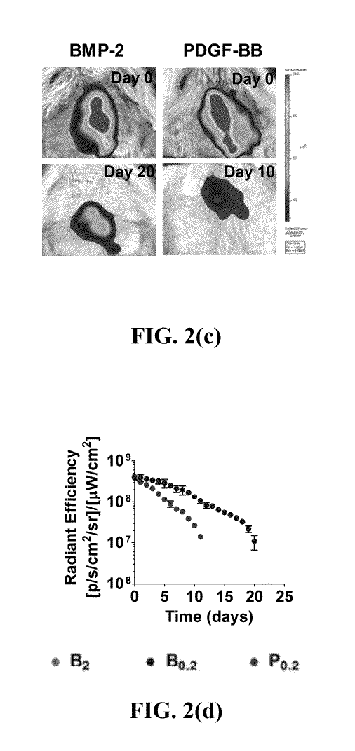

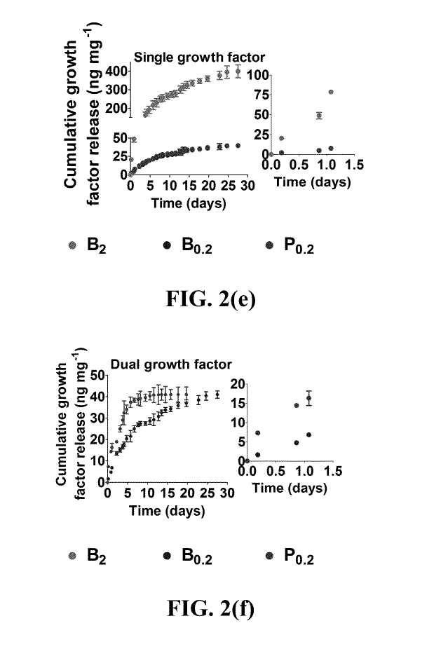

A relevant model to illustrate the clinical translational potential for treating CMF bone defects is a critical-size calvarial defect in a skeletally mature rat, corresponding to an 8 millimeters circular wound. Calvarial defects can answer questions about the biocompatibility and the biological functions of bone repair materials and morphogens before putting them into a clinical setting. The healing process in this animal model has been well characterized. In addition the 8 millimeter defect size is too large to heal without intervention. It has been demonstrated that the rate of scaffold degradation is critical to bone healing. The kinetics of degradation of the PLGA membrane in the wound healing environment of the defect were examined first. A thickness of a porous polymer membrane was held constant at 120±10 microns as measured by SEM and through monitoring of in vivo degradation as a function of the PLA:PGA ratio in the PLGA copolymer. The objective was to select a ratio that would yield a degradation half-life of about 4 weeks to coincide with bone growth. The mass and diameter of the uncoated membranes placed in the rat cranial defect were monitored at pre-determined time intervals to determine a relationship between copolymer ratio and rate of degradation. FIGS. 2(a)-(b) show degradation profiles of the PLGA membrane in a rat calvaria as a function of a PLA:PGA ratio. Degradation was measured by dry mass difference and change in diameter. Data represent the means±s.e.m., n=4 per group per time point. As anticipated, PLGA bulk eroded at all PLA:PGA ratios as indicated by the gradual decrease in dry weight. It was observed that PLA:PGA (50:50) yielded a desirable degradation profile for cranial defect healing with a degradation of half-life of about 4 weeks. As such, PLA:PGA (50:50) was selected for further evaluation. End-functionalization of alendronate to the PLGA (50:50) backbone did not noticeably alter the in vivo degradation kinetics. Each implant was about 5 mg, and the dose of alendronate per implant was ˜10 μg.

Progenitor cell activation and bone tissue repair are highly sensitive to growth factor dose and its local availability. To induce the desired biological response for bone tissue repair, we examined the effect of growth factor combinations released from the PEM film. To maintain film thickness and duration of biologic release across the different combinations, we aimed to incorporate different amounts of growth factor with the same number of layers in the film. 40 layers of each growth factor were applied either individually or in combination in a B or P tetralayer repeat unit. Drug loading per layer was proportional to growth factor concentration and was used to control the amount of growth factor that was incorporated in the PEM film. In dual growth factor releasing PEMs, the growth factors were arranged so that BMP-2 was incorporated in the bottom about 40 layers closest to the membrane and the PDGF-BB was incorporated in the subsequent about 40 layers. BMP-2 and PDGF-BB concentration was tracked using near-IR dyes in the same animal. FIG. 2(c) is an illustration of the concentration gradient of the growth factors over time in a rat, as detected by near-IR dyes. FIG. 2(d) is a plot of Radiant Efficiency measured versus Time (in days) of growth factors administered to an animal by an embodiment of the invention. As shown by the plot of FIG. 2(d), the two growth factors, BMP-2 and PDGF-BB, are sustained over different times, with the PDGF-BB being detectable for about 11 days after surgery, and BMP-2 detectable for 20 days. FIGS. 2(e)-(f) show in vitro growth factor release in single and combination films from PLGA membranes. FIG. 2(e) is a plot of single growth factors growth over time. FIG. 2(f) is a plot of dual growth factor growth over time. Data represent the means±s.e.m., n=6 per group per time point. This resulted in a concentration gradient of growth factors within the film and allowed for differential rates of growth factor release with complete elution of PDGF-BB followed by BMP-2 in cell culture release media. Very similar release profiles were observed from the P and B single growth factor films. The sequence of release is consistent with the recapitulation of a natural bone wound healing cascade, in which osteogenesis typically follows vascularization. Importantly, burst release of either growth factor was not observed; rather the release was sustained over different times, as intended. In vitro, approximately 20% of growth factor from the single factor PEM eluted within approximately 24 hours after release. Within this 24 hour time period, the release rate is approximately constant in this time period (R2=0.951). In vivo, we observed a decrease in the fluorescence signal of approximately 22% and 6% for the PDGF-BB and BMP-2 respectively over the same time period. The release reported in this study is an order of magnitude lower than what has typically been reported for single growth factor burst release systems, in which 40-60% of the growth factor is released within 3 hours after release, with low therapeutic effect.

### Bone Repair in a Rodent Calvaria Critical Size Defect

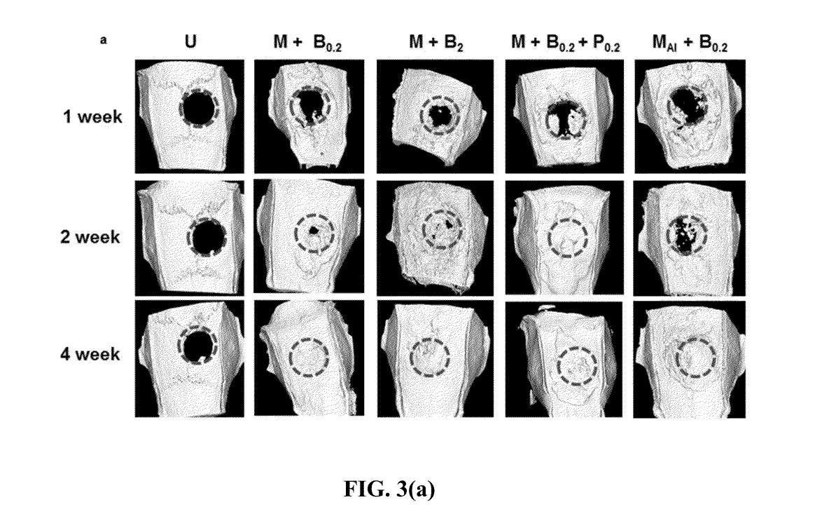

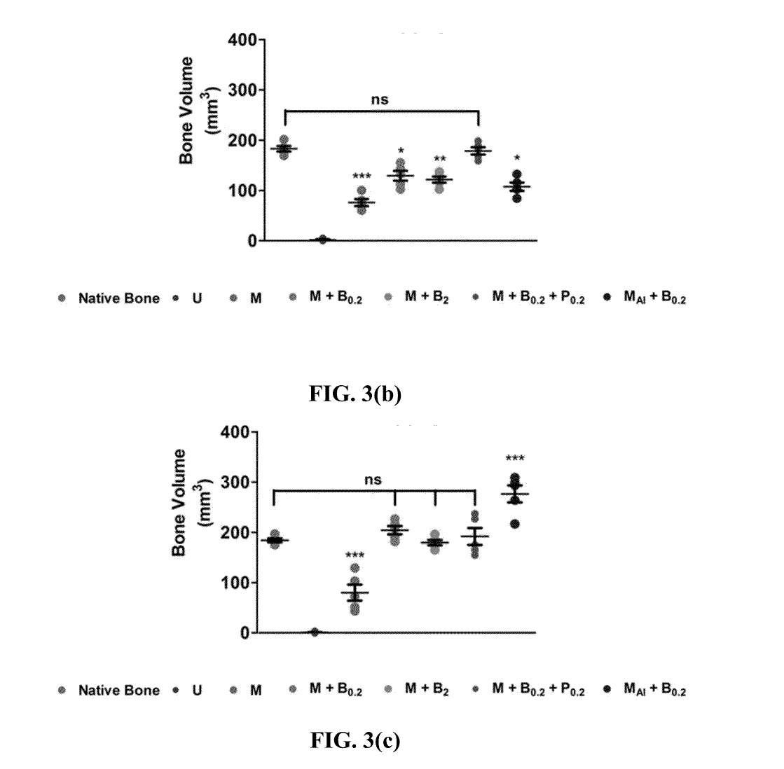

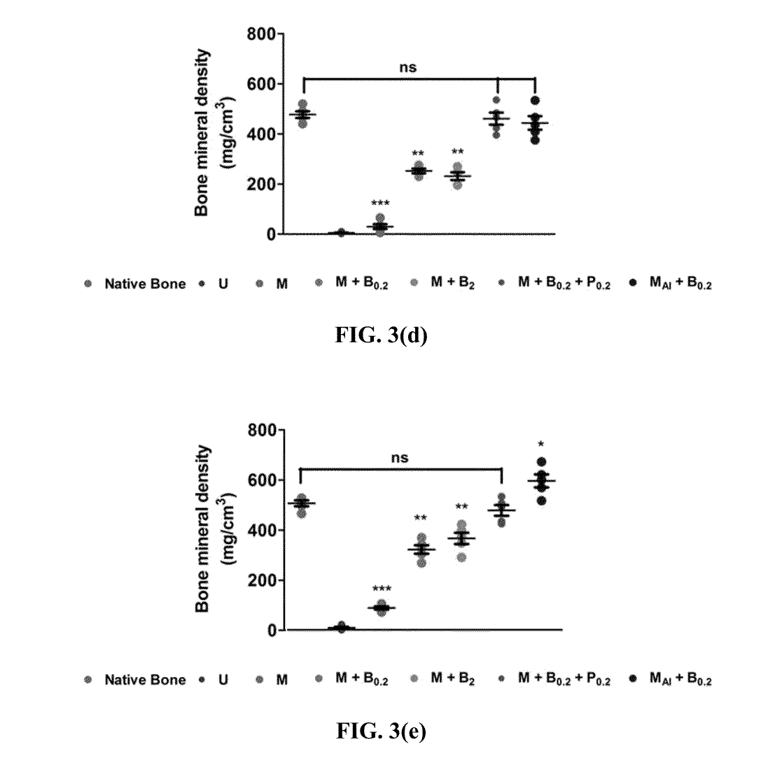

The effect of growth factor formulations on inducing tissue repair is shown in Table 1. Bone healing in this model is characterized by new bone tissue deposition and coverage of the defects. The healing process was temporally monitored using microcomputed tomography (μCT). FIG. 3(a) shows representative radiographs of bone formation around drilled implants with different films at 1, 2, and 4 weeks. The broken circle indicates the location of the defect in each radiograph and has an 8 millimeter diameter. Defect closure was achieved in all animal groups with different treatment conditions within 4 weeks. n=5 per group. As anticipated, no bone healing was observed in an untreated defect. Spicules of bone were observed with an uncoated membrane. A PEM coated membrane with B and P+B layers induced a potent bone healing response and induced closure within 4 weeks post-treatment. Defects reconstructed with growth factor associated PLGA membranes exhibited multifocal bone formation, where new bone formation initiated at the margins and gradually filled in the defect.

TABLE 1
Effect of growth factor formulations on inducing tissue repair

| | Experimental Group | Description |
|---|---|---|
| 1 | Untreated (U) | Untreated defect |
| 2 | PLGA (50:50) membrane only (M) | Defect treated with an |
| | | uncoated membrane |
| 3 | PLGA Membrane + BMP-2 (0.2 micrograms) (M + B_{0.2}) | Membrane coated with low |
| | | dose BMP |
| 5 | PLGA Membrane + BMP-2 (2 micrograms) (M + B₂) | Membrane coated with |
| | | high dose BMP |
| 4 | PLGA Membrane + BMP-2 (0.2 micrograms) + PDGF-BB | Membrane coated with low |
| | (0.2 micrograms) | dose BMP and low dose |
| | (M + B_{0.2} + P_{0.2}) | PDGF |
| 6 | PLGA-Alendronate Membrane + BMP-2 (0.2 micrograms) | PLGA-Alendronate |
| | (M_{Al} + B_{0.2}) | membrane coated with low |
| | | dose BMP |

Images in FIG. 3(a) were used to quantify bone volume and bone mineral density at 2 and 4 weeks within the regions of interest marked by dotted red circles. Each point represents individual animal. Data are means±s.e.m. (n=5-6 per group) *p<0.05, **p<0.01, ***p<0.001, ns=not significant, ANOVA with Tukey post hoc test. All groups are compared with the mechanical properties of the M+B_{0.2}+P_{0.2} group. As shown by FIGS. 3(b)-(e), repair initiated by P+B layers together resulted in a smaller defect after 2 weeks (FIGS. 3(b) and 3(d)) compared to single factor BMP-2 induced repair. While increasing the total dose of BMP-2 above 0.2 micrograms did not appear to alter the rate of bone repair, a qualitative comparison of the two groups at 2 weeks indicated that more BMP-2 resulted in a greater level of bone remodeling activity at and around the defect site. Using an M_{Al} porous polymer membrane resulted in a remarkable difference to the rate and quality of bone repair. At 2 weeks, single growth factor BMP-2 release from the M_{Al} membrane appeared to reduce the rate of bone repair, and resulted in a larger defect when compared to the unmodified membrane, likely owing to the inhibition of bone remodeling and migration of new bone into the defect. However, at the end of 4 weeks (FIGS. 3(c) and 3(e)), the defect completely bridged with new bone that had a significantly higher bone volume (BV) and bone mineral density (BMD) than the single and dual growth factor groups. Taken together, these observations suggest that the alendronate binds with high affinity to newly formed bone tissue to prevent rapid remodeling by inhibition of osteoclast activity, a known physiological effect of bisphosphonates. The action of BMP-2 causes osteoblasts to continue bone deposition, thus significantly more bone tissue is present throughout the repair site. These observations are consistent with the known mechanism of bisphosphonate action. At 4 weeks, the BMD of bone formed by B layers alone was lower than that of native calvaria and bone formed by P+B layers. However, these groups had comparable BV, suggesting that BMP-2 delivery alone resulted in less mature bone.

### Histological Examination of Regenerated Bone

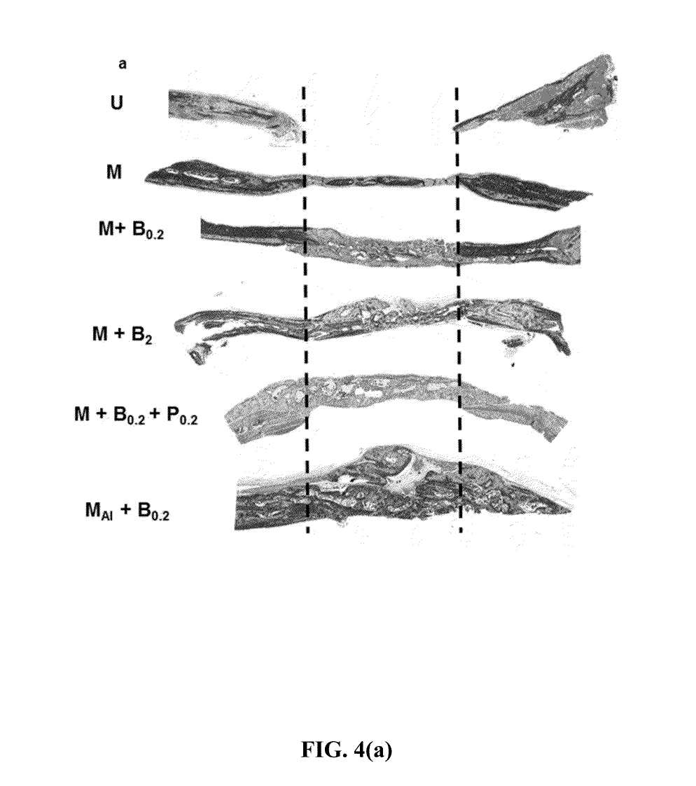

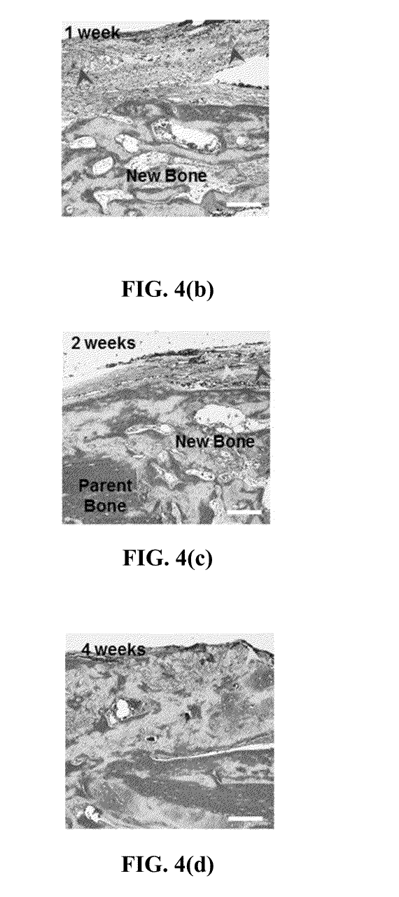

FIG. 4 illustrates histology of new tissue formed with various film formulations. FIG. 4(a) shows each image as a cross section of the calvarial defect after 4 weeks, at which time different levels of bone tissue morphogenesis was observed at the defect site. The broken lines indicate the position of the defect site and are 8 millimeters apart. Collagen is represented by blue and osteocytes (mature bone) is represented by red. Sections were stained with Masson's trichrome stain and viewed under bright field microscopy. A histological examination revealed the underlying cellular processes involved in bone repair. There were no indications of adverse foreign body reactions as evidenced by the lack of foreign body giant cells, long-term inflammation or infection. Bone formation processes were completely absent in the untreated defect. Tissue formation in the uncoated membrane group showed collagen fibers present with partial bony ingrowth at the wound margins. Outer and inner cortical tables were variably present. In contrast, bone formed under the influence of growth factors in the treatment groups was trabecular, with evidence of remodeling and maturation with extensive bone development in a hypercellular environment that is characteristic of bone wound healing. In all growth factor treated groups, the defect was completely bridged within 4 weeks with bone that exhibited ongoing active remodeling processes for all growth factor treated groups. New bone formed as a result of B layers alone lacked mineralization and compact bone formation. The osteoid layer had wide borders indicating that rapid tissue deposition preceded mineralization. Qualitatively, the bone formed by P+B layers had a greater number of vascular channels and a higher cell density within the bone, indicating the mitogenic role of PDGF-BB in the bone formation process.

Growth factor coated PLGA polymer membrane resulted in bone repair via intramembranous ossification preceded by highly cellular granulation tissue supported by the membrane. FIGS. 4(b)-(d) shows granulation tissue layer at 1 (FIG. 4(b)), 2 (FIG. 4(c)) and 4 (FIG. 4(d)) weeks during bone repair in the M+B_{0.2}+P_{0.2} treatment group. The tissue gradually reduces in thickness from 1 to 4 weeks as bone repair is completed. Pieces of the PLGA membrane were observed in some section (scale bar, 30 microns). Arrows: PLGA membrane; granulation tissue layer. As new bone filled the gap, it was observed that the tissue layer remodeled and reduced in thickness from 1 to 2 weeks, eventually reducing to a one-cell thick layer form after bone had completely filled the gap at about 4 weeks post-surgery. The thick tissue layer was a rich source of progenitor cells for bone repair and helped nucleate the repair machinery. Bone formation under the influence of the M_{Al} membrane bridged the gap with excess bone that lacked specific orientation and was less compact compared to bone formed under the influence of B layers alone. These observations are consistent with a lack of remodeling behavior in the presence of alendronate.

### Comparison of Bone Mechanical Properties

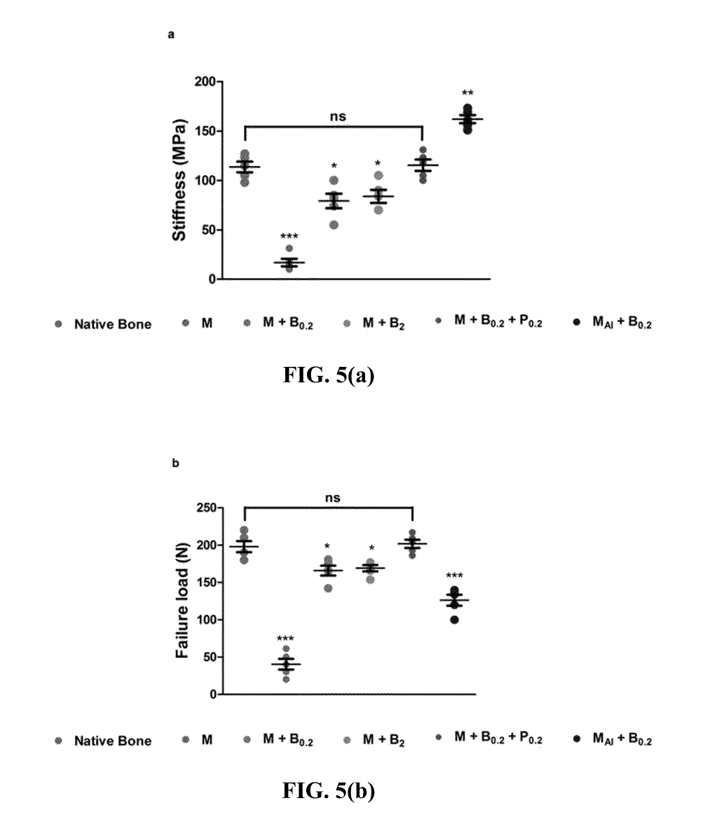

Compression tests were performed to investigate the mechanical integrity of the reconstructed region and obtain a measure of the mechanical properties of the restored bone. Stiffness and compressive failure force for the regenerated bone for the different groups at the 4 week end-point were measured and compared to native calvaria bone that was not injured. FIG. 5(a) shows stiffness and FIG. 5(b) shows failure load from different groups at 4 weeks after implantation. Data are means±s.e.m, (n=5 implants per group), *p<0.05; **p<0.01; ***p<0.001, ns=not significant, ANOVA with a Tukey post hoc test. All groups are compared with the mechanical properties of the M+B_{0.2}+P_{0.2} group. Tissue regenerated with the uncoated PLGA membrane had lower stiffness and resistance of 16.9±3.8 (s.e.m.) MPa to compressive load. Bone formation was significant, organized and cohesive with B layers alone and thus had a higher stiffness of ˜82 MPa, independent of BMP-2 dose. This value was approximately 27% lower than the stiffness of native calvaria bone. However, bone formation with P+B layers was comparable to that of native bone. These observations correspond well to the disparate histological observations. Bone formed with B layers alone was less mature, lacked significant mineralization, which resulted in a lower stiffness compared to native bone. On the other hand, PDGF-BB and BMP-2 co-delivery resulted in mature bone formation with mechanical properties identical to native calvaria. A similar structure-property relationship existed for bone formed with the M_{Al} membrane. As noted, the excess bone present was not compact and we observed that the bone was approximately 43% stiffer than the native calvarial bone. Compressive failure loads were also compared with native calvarial bone. Tissue formed by uncoated PLGA membrane had reduced resistance to compressive loads. Bone formed by BMP-2 alone had approximately 14% lower compressive strength than native calvaria bone and was dose independent, owing to a lack of maturation and corresponded with the observation of lower stiffness. BMP-2 and PDGF-BB acted in concert to induce bone with the same mechanical loading behavior as that of the native calvaria. Interestingly, while the M_{Al} membrane resulted in stiffer bone, the mechanical failure load was significantly lower. This too is explained by the lack of compact bone formation, which resulted in brittle bone formation that was not cohesive and thus unable to distribute load uniformly. In all groups, the PLGA membrane was designed to degrade after bone repair and as such not expected to affect the strength.

The search for new bone regeneration strategies in particular is a key priority fueled by the increasing medical and socioeconomic challenge of an aging population. In this study, we have used materials for directing bone tissue repair processes by the fine-tuned and robust tunable spatio-temporal control of biologics from a thin film, an approach that could be a key development for next-generation biomedical devices. Previous work has demonstrated the benefit of delivering multiple growth factors for bone tissue engineering. Typically, growth factors are released from particle systems or scaffolds that persist in the wound and in some cases may even hinder formation of cohesive, mechanically competent bone that also recapitulates geometry. Often, the new bone may not be adequately vascularized which hinders remodeling and integration. The time taken to induce repair is significantly longer and the reported bone strength with these permanent systems is often lower than native bone. The present studies suggest that tailoring the degradation of the delivery vehicle, recapitulation of the natural healing cascade mediated by the release of specific growth factors and forming a granulation tissue layer that supplies progenitor cells that differentiate are critical to the enhanced bone regeneration. A strategy of delivering multiple growth factors with tunable control is particularly crucial in higher order animals including humans, which have a slower rate of bone repair than rodents. By adding specific materials known to play a role in bone formation, a rate, amount and quality of bone repair was controlled and structure-function relationships on a molecular level were provided.

Large scale production of proposed bone repair materials is a significant barrier to its clinical application and has severely limited its translational potential. Our method of making composite devices is scalable. This is evidenced by the manufacture of water filtration membranes and coated surfaces that can be several meters long and of controllable thickness. In prior LbL work, the polymer layers are incubated in a concentrated solution of a single growth factor for an extended period of time. Typically, such systems exhibit a burst release profile, in which much of the therapeutic is ejected from the LbL films very quickly (>80% in less than 24 hours). The importance of controlling the release of biologics from multilayer films has been previously demonstrated. Importantly, PEM assembly uses mild, aqueous conditions that preserve the activity of fragile biologics. Concurrently, the approach of developing tissue engineered constructs in vitro for the purpose of in vivo transfer remains limited by the amount of vascularization of the graft. Our studies suggest that introducing controlled interconnected gradient porosity into a cell-free material system can recapitulate an intramembranous bone formation process with the creation of a vascular network to augment bone formation. Lack of toxicity is critical for materials used in implantable devices, and the long-term host response to permanent implants continues to be a concern. In this work all the components were selected with biocompatibility in mind: PLGA, used as the polymer support film, is a bioresorbable polymer with a long history of clinical use in drug delivery devices; furthermore, the same approach can be applied to other biodegradable membranes and scaffolds. To coincide with bone healing, the PLGA membrane degraded over several weeks, allowing for transport of the breakdown products away from the implant site. In fact, PLGA is clinically used in bone fixation systems for the cranium. Previous studies have demonstrated the compatibility of the poly(β-amino ester) family in vitro and in vivo. PAA is a well-characterized weak polyanion with a high charge density distributed over a non-erodible backbone that has been listed as an approved excipient in the FDA's Inactive Ingredient Guide and is used in the clinic. Consistent with these expectations, we observed no local toxicity in any of the animals treated throughout these studies. It has been demonstrated that this formulation of growth factors in multilayer films on devices maintains their bioactivity when stored at room temperature in the dry state, potentially alleviating the need for refrigeration during the distribution and storage. Composite devices can be customized on demand by a surgeon to induce repair in a variety of bone defects. Although the true potential of any bone regeneration strategy can only be realized through large animal pre-clinical studies and ultimately human clinical trials, the data shown here suggest that bone healing using an engineered regenerative surface is a potent strategy for safe, precise and targeted tissue repair, and a platform technology with the potential to be applied universally in regenerative medicine.

Dose tunability and delivery of these potent biologics in a manner that can be adapted for clinical application is critical to success of this strategy. The release rates of the growth factors can be tailored using the PEM coatings. Typically, PEM coatings have characteristics of both a stratified and blended film. There is a concentration gradient of materials in the film, in the order that they are deposited. In these films, the BMP-2 is enriched in bottom layers of the film and the PDGF-BB in the top. When the film surface degrades from the top-down, the growth factor factors elute, in which the PDGF-BB elutes faster than the BMP-2. In addition, the pores in the membrane provide an additional means to sequester the BMP-2 enriched PEM coating—further contributing to a more sustained release. Particle systems or scaffolds that persist in the wound, in some cases, may even hinder formation of cohesive, mechanically competent bone that also recapitulates geometry. The time taken to induce repair is significantly longer and the reported bone strength with these permanent systems is often lower than native bone. The release of specific known growth factors, BMP-2 and PDGF-BB either individually or in combination, is critical to enhanced bone regeneration. This combination of growth factors has been reported to induce rapid and successful bone tissue regeneration (29). Both PDGF and BMP-2 are growth factors that participate in the bone healing cascade. It is known that introducing PDGF expands the number of progenitor cells available to induce bone repair. Therefore, an early, sustained signal of this growth factor has a directly increases the rate of repair, at levels that could not be achieved even by 10-fold increase in the dose of BMP-2. This strategy of delivering multiple growth factors with tunable control is particularly crucial in higher order animals with slower rates of bone repair, including humans. The PEM coating can be applied even if the membrane itself were modified to tune the degradation kinetics for adoption to higher animals.

The composite PEM coating can be scaled to complex surfaces with large dimensions. Importantly, PEM assembly uses mild, aqueous conditions that preserve the activity of fragile biologics. Lack of toxicity is critical for materials used in implantable devices, and the long-term host response to permanent implants continues to be a concern. Components were selected with biocompatibility in mind: PLGA is a biodegradable polymer with a long history of clinical use in drug delivery devices and used in bone fixation systems with no adverse immunogenic responses. The surface of the PLGA membrane with the smaller pores and lower porosity (polymer dense) surface faced outward, towards the skin. The different pore sizes on the PLGA membrane surface were used to (i) form a temporary barrier with nanoscale pores and prevent soft tissue prolapse into the wound, (ii) allow progenitor cell infiltration in the less polymer dense, microporous surface and (iii) achieve adaptable, controlled growth factor release. The membrane remained intact and structurally competent over the timescale of bone formation. The use of the PEM was essential in this example, as the uncoated micro-porous membrane resulted in the formation of a fibrous tissue layer. Furthermore, the same approach with PEM coatings can be applied to other biodegradable membranes and scaffolds, as we have described previously. PAA is a well-characterized weak polyanion with a high charge density distributed over a non-erodible backbone that has been listed as an approved excipient in the FDA's Inactive Ingredient Guide in oral and topical drug delivery formulations. Therefore, there is a path to regulatory approval for its use in a degradable implant. The amount of alendronate (˜10 μg/implant) is several orders of magnitude lower than the doses that are known to cause side-effects. Consistent with these expectations, no local toxicity in any of the animals treated throughout these studies. Importantly, this strategy is cell-free and does not rely on the extraction and ex-vivo expansion of progenitor cells for re-implantation in the body. In effect, these nanolayered coatings can be adapted on demand to induce repair in a variety of bone defect types by recruiting endogenous progenitor cells. This approach provides a new alternative to autologous bone grafts for CMF bone repair and reconstruction. Although the true potential of any bone regeneration strategy can only be realized through large animal pre-clinical studies and ultimately human clinical trials, the data shown here suggest that bone healing using an engineered regenerative surface is a potent strategy for safe, precise and targeted tissue repair, and demonstrates the use of alternating nanolayer assembly as a platform technology with the potential to be applied universally in regenerative medicine.

### Uses

In some embodiments, provided composite devices are administered or implanted using methods known in the art, including invasive, surgical, minimally invasive and non-surgical procedures, depending on the subject, target sites, and agent(s) to be delivered.

In some embodiments, the present invention presumes a plurality of different growth factors (e.g., BMP or PDGF), each of which is directed to a different defect. The present invention encompasses a recognition that the described technology permits facile and close control of relative amounts of such different growth factors that are or can be delivered to a defect site. The present invention encompasses a recognition that growth factor can be designed and/or prepared to controllably deliver to a defect site over a period. The present invention also encompasses a recognition that rapid resolution of the defect is desirable and different types of defects respond to different growth factor, which may be temporally dose dependent.

The present invention demonstrates a synthesis of composite devices including a porous polymer membrane associated with growth factor for controllable delivery of growth factor to a bone defect site.

The present invention demonstrates a synthesis of composite devices including a porous polymer membrane associated with growth factor and LbL film associated with growth factor for controllable delivery of growth factor to a bone defect site. In some aspects, the present invention specifically encompasses the recognition that LbL assembly may be particularly useful for coating a porous polymer membrane described herein. There are several advantages to coat porous polymer membranes using LbL assembly techniques including mild aqueous processing conditions (which may allow preservation of biomolecule function); nanometer-scale conformal film of surfaces; and the flexibility to coat objects of any size, shape or surface chemistry, leading to versatility in design options. According to the present disclosure, one or more LbL films can be assembled on and/or associated with a porous polymer membrane. In some embodiments, a porous polymer membrane having one or more growth factors associated with the LbL film, such that decomposition of layers of the LbL films results in release of the growth factors. In some embodiments, assembly of an LbL film may involve one or a series of dip coating steps in which a core is dipped in coating solutions. Additionally or alternatively, it will be appreciated that film assembly may also be achieved by spray coating, dip coating, brush coating, roll coating, spin casting, or combinations of any of these techniques.

The present invention encompasses a recognition that the composite and methods disclosed herein are suited for craniomaxillofacial reconstruction. The present invention encompasses a recognition that repair of large bone defects includes, for example, the skull, calvaria, jaw, or long bones. The present invention encompasses a recognition that repair includes Mandibular/Maxillary augmentation for dental implants. The present invention encompasses a recognition that repair of defects through isolating and delivery of growth factor as described herein to a cell, tissue, or organ of a subject. Examples of target sites include but are not limited to the bone, eye, blood vessels, pancreas, kidney, liver, stomach, muscle, heart, lungs, lymphatic system, thyroid gland, pituitary gland, ovaries, prostate, skin, endocrine glands, ear, breast, urinary tract, nervous tissue, brain matter or any other site in a subject. The present invention further encompasses bone repair to sites subject to disease, disorder or condition, for example, osteosarcoma.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

1. A composite device for controlled formation of tissue, comprising:
a porous polymer membrane that degrades, decomposes, and/or delaminates when placed in a physiological environment; and
at least one growth factor,
the device being arranged and constructed so that the at least one growth factor is released from the device over time after the device is placed in the physiological environment.

2. The composite device of claim 1, wherein the porous polymer membrane comprises the growth factor, and the growth factor is released as the porous polymer membrane degrades, decomposes and/or delaminates.

3. The composite device of claim 2, wherein the porous polymer membrane has a thickness of at least about 5 microns to at least about 200 microns.

4. The composite device of claim 3, wherein the porous polymer membrane comprises a plurality of interconnected pores.

5. The composite device of claim 4, wherein the porous polymer membrane has uniform porosity.

6. The composite device of claim 5, wherein a pore size of the plurality of interconnected pores is about the same between a top surface of the porous polymer membrane and a bottom surface of the porous polymer membrane.

7. The composite device of claim 4, wherein the porous polymer membrane has non-uniform porosity.

8. The composite device of claim 7, wherein a size of a pore of the plurality of interconnected pores varies between a top surface of the porous polymer membrane and a bottom surface of the porous polymer membrane.

9. The composite device of claim 8, wherein the pore size increases between the top surface of the porous polymer membrane and the bottom surface of the porous polymer membrane.

10. The composite device of claim 9, wherein the pore size varies from 200 nanometers on the top surface of the porous polymer membrane to 2 millimeters on the bottom surface of the porous polymer membrane.

11. The composite device of claim 9, wherein the pore size varies from 2 microns on the top surface of the porous polymer membrane to 200 microns on the bottom surface of the porous polymer membrane.

12. The composite device of claim 8, wherein the porous polymer membrane is bifunctional.

13. The composite device of claim 12, wherein the top surface of the porous polymer membrane is impermeable and the bottom surface of the porous polymer membrane is permeable.

14. The composite device of claim 4, wherein the porous polymer membrane is comprised of polycaprolactone (PCL).

15. (canceled)

16. The composite device of claim 4, wherein the porous polymer membrane is comprised of poly(glycolide-colactide) copolymer (PLGA)/polylactic acid (PLA).

17-21. (canceled)

22. The composite device of claim 16, wherein the porous polymer membrane comprises PLA and PGLA in a 50:50 ratio by weight.

23. The composite device of claim 16, wherein the porous polymer membrane further comprises a bisphosphonate conjugated to the PLGA.

24. The composite device of claim 23, wherein the bisphosphonate is alendronate.

25. The composite device of claim 1, wherein the porous polymer membrane further comprises a dopant.

26. The composite device of claim 25, wherein the dopant is PVP (polyvinylpyrrolidone) or PEO (polyethylene oxide).

27. The composite device of claim 26, wherein the porosity of the porous polymer membrane varies with the dopant.

28. The composite device of claim 1, further comprising a multilayer film associated with the membrane.

29. The composite device of claim 28, wherein the multilayer film comprises a layer-by-layer (LbL) film.

30. The composite device of claim 29, wherein the LbL film is hydrolytically degradable.

31. The composite device of claim 30, wherein the LbL film has a thickness of at least about 100 nanometers to at least about 1 micron.

32. The composite device of claim 31, wherein the LbL film comprises alternating polyelectrolyte multilayers (PEM), wherein adjacent layers of the LbL film are associated with one another via one or more non-covalent interactions.

33. The composite device of claim 32, wherein the multilayer film comprises the growth factor, and wherein the growth factor is associated within the alternating PEM of the LbL film.

34. (canceled)

35. The composite device of claim 33, wherein a loading dose of the growth factor is at least about 10 nanograms to at least about 10 micrograms.

36. The composite device of claim 33, wherein the LbL film decomposes, degrades and/or delaminates releasing the growth factor.

37. The composite device of claim 36, wherein growth factor is released with a rate of at least about 1 nanogram to at least about 20 nanograms of the growth factor per milligram of membrane per day.

38. The composite device of claim 37, wherein release of the growth factor occurs over at least about 2 days to at least about 30 days.

39. The composite device of claim 33, wherein the LbL film comprises at least one growth factor.

40-43. (canceled)

44. The composite of claim 36, wherein the LbL film degrades, decomposes, and or delaminates with a concurrent release of the growth factor.

45. The composite of claim 36, wherein the LbL film degrades, decomposes, and or delaminates with a staggered release of the growth factor.

46. The composite device of claim 1, wherein the growth factor is a bone morphogenetic protein (BMP), a platelet-derived growth factor (PDGF), a vascular endothelial growth factor (VEGF), and/or placental growth factor (PIGF).

47. The composite device of claim 33, wherein the growth factor is a bone morphogenetic protein (BMP), a platelet-derived growth factor (PDGF), a vascular endothelial growth factor (VEGF), and/or placental growth factor (PIGF).

48. The composite device of claim 46, wherein the LbL film has a tetralayer repeat unit of [Poly2/PAA/PDGF/PAA].

49. The composite device of claim 46, wherein the LbL film has a tetralayer repeat unit of [Poly2/PAA/BMP/PAA].

50. The composite of claim 47, wherein the LbL film comprises a tetralayer repeat unit of [Poly2/PAA/BMP/PAA] comprising 40 layers closest to the porous polymer membrane and a tetralayer repeat unit of [Poly2/PAA/PDGF/PAA] comprising 40 layers subsequent to the 40 layers closest to the porous polymer membrane.

51. The composite of claim 50, wherein the LbL film degrades releasing PDGF followed by BMP.

52. The composite of claim 51, wherein the LbL film degrades quickly releasing PDGF at the rate of at least about 4 nanograms growth factor per milligram of membrane per day, followed by a sustained release of BMP at the rate of at least about 1 nanograms growth factor per milligram of membrane per day.

53. The composite of claim 52, wherein the LbL film degrades with a concurrent release of PDGF and BMP.

54. The composite device of claim 1, wherein the porous polymer membrane covers, fills, and/or isolates a defect when a shape of the porous polymer membrane replicates a shape of the defect and/or a size of the porous polymer membrane is at least a size of the defect.

55. The composite device of claim 54, wherein the defect is a large bone defect.

56. The composite device of claim 55, wherein the large bone defect is a craniomaxillofacial (CMF) defect.

57. The composite device of claim 55, wherein the defect is a vascular tissue defect.

58. The composite device of claim 55, wherein the defect is a neural tissue defect.

59-60. (canceled)

61. The composite device of claim 55, wherein growth factor is controllably released following degradation of the porous polymer membrane when the membrane is placed in contact with the defect in an environment under physiological conditions.

62-70. (canceled)

71. The composite device of claim 1, wherein the porous polymer membrane is biocompatible, biodegradable, and/or resorbable.

72. The composite device of claim 71, wherein the porous polymer membrane has a thickness of at least about 5 microns to at least about 200 microns.

73-85. (canceled)

86. The composite device of claim 1, wherein the porous polymer membrane comprises an agent to be delivered, and the agent to be delivered is released as the porous polymer membrane degrades, decomposes and/or delaminates.

87. The composite device of claim 86, wherein the agent is a small molecule.

88. The composite device of claim 87, wherein the small molecule is an antibiotic.

89. The composite device of claim 88, wherein the antibiotic is gentamicin.

90. The composite device of claim 1, wherein the tissue is bone.

91. The composite device of claim 1, wherein the porous polymer membrane comprises a first agent to be delivered and a second agent to be delivered, and the first and second agents to be delivered are released as the porous polymer membrane degrades, decomposes and/or delaminates.

92. The composite device of claim 91, wherein the first agent and the second agent are each small molecules.

93. The composite device of claim 92, wherein the first small molecule is an antibiotic.

94. The composite device of claim 93, wherein the antibiotic is gentamicin.

95. The composite device of claim 94, wherein the second small molecule is an anti-inflammatory agent.

96. The composite device of claim 95, wherein the anti-inflammatory agent is ibuprofen.
